(19) United States
(12) Patent Application Publication
(10) Pub. No.: US 2013/0190726 A1
(43) Pub. Date: Jul. 25, 2013

(54) MOTION COMPENSATING CATHETER DEVICE

(71) Applicants: Samuel Kesner, Somerville, MA (US); Robert Howe, Cambridge, MA (US); Shelton Yuen, Berkeley, CA (US); Pedro Del Nido, Lexington, MA (US); Douglas Perrin, Cambridge, MA (US); Nikolay Vasilyev, Newton, MA (US)

(72) Inventors: Samuel Kesner, Somerville, MA (US); Robert Howe, Cambridge, MA (US); Shelton Yuen, Berkeley, CA (US); Pedro Del Nido, Lexington, MA (US); Douglas Perrin, Cambridge, MA (US); Nikolay Vasilyev, Newton, MA (US)

(73) Assignees: CHILDREN'S MEDICAL CENTER CORPORATION, Boston, MA (US); PRESIDENT AND FELLOWS OF HARVARD COLLEGE, Cambridge, MA (US)

(21) Appl. No.: 13/643,625

(22) PCT Filed: Apr. 29, 2011
(86) PCT No.: PCT/US11/34544

§ 371 Date: Mar. 20, 2013

Related U.S. Application Data
(60) Provisional application No. 61/329,803, filed on Apr. 30, 2010.
(60) Provisional application No. 61/388,813, filed on Oct. 1, 2010.

(51) Int. Cl. A61M 25/01

(52) U.S. Cl. CPC A61M 25/0116
USPC 604/510; 604/95.01

(57) ABSTRACT

A catheter system allows for percutaneous intervention on the fast-moving structures inside the heart. The device consists of a steerable catheter sheath, an actuated, multi-degree of freedom moving catheter, and a catheter end effector, such as a fixation device. An actuator controls the motion of the catheter guide-wire in the catheter sheath to follow the motion of the target tissue. A control system controls the actuator and compensates for mechanical characteristics of the system including friction and backlash. A 3-D imaging system can be used to view the motion of the target tissue and the catheter end effector and produce 3-D imaging data. The 3-D imaging data can be used by the control system to track the target tissue and accurately position the end effector with respect to the moving target tissue allowing a clinician to repair the target tissue while it is moving. The system can be used to perform mitral valve annuloplasty wherein the actuated catheter compensates for the heart valve motion and an end effector fixation device is used to place fixation items, like anchors or staples, into the heart valve tissue to fix the annulus.

### CROSS-REFERENCE TO RELATED APPLICATIONS

This application claims any and all benefits as provided by law of U.S. Provisional Application Nos. 61/329,803 filed 30 Apr. 2010 and 61/388,813 filed 1 Oct. 2010, which are hereby incorporated by reference in their entirety.

### STATEMENT REGARDING FEDERALLY SPONSORED RESEARCH

This invention was made with government support under HL073647 awarded by the National Institutes of Health. The government has certain rights in this invention.

### REFERENCE TO MICROFICHE APPENDIX

Not Applicable

### BACKGROUND

1. Technical Field of the Invention

The present invention is directed to a catheter device for use in percutaneous surgery. Specifically, the invention is direct to a motion compensated catheter device for use in surgery on fast moving structures of the body, such as a beating heart.

2. Description of the Prior Art

Catheter technology has greatly expanded the range of surgical procedures that can be performed with minimal intrusion into the body. Traditional surgical procedures are very invasive involving large incisions and the displacement of the soft tissue around the organ or area to be operated on. Many of these procedures have been replaced with minimally invasive catheter base procedures.

For example, interventional cardiologists can now perform many inside the heart procedures using minimally invasive techniques. These procedures include measuring cardiac physiological function, dilating vessels and valves, and implanting prosthetics and devices. Nonetheless, current catheter technologies do not allow clinicians to interact with beating heart tissue in order to order to perform procedures that require open heart surgery.

Cardiac catheters are long and thin flexible tubes that are inserted into the vascular system and passed into the heart. Current robotic cardiac catheters, such as the commercially available Artisan Control Catheter (Hansen Medical, Mountain View Calif., USA), permit a human operator to control the positioning of a catheter in the lateral direction and advance it through the vasculature. However, these systems do not provide sufficient speeds to compensate for the motion of the beating heart.

Researchers have developed robotic approaches to compensating for the motion of the beating heart, but these techniques are directed at coronary artery bypass procedures that repair arteries on the external heart surface. In previous work, we developed robotic devices that compensate for the motion of internal heart structures with a handheld robotic instrument inserted through incisions in the heart wall. This work shows that single degree of freedom (DOF) servoing is sufficient to accurately track the motion of certain cardiac structures, including the human mitral valve annulus. This approach alleviates the risks associated with stopped heart techniques, but the necessity of creating incisions in the heart wall means that this approach is not minimally invasive.

### SUMMARY

This present invention is directed to a catheter that employs motion compensation techniques and optionally, other corrections in order to minimize invasiveness in surgical procedures involving moving target areas. In accordance with one embodiment of the invention, the system controls an actuator at the base of the catheter system that can drive a catheter guide-wire inside a flexible catheter sheath to follow the motion of the moving target tissue. For example, the sheath can be manually or automatically advanced through the vasculature into the heart. A sensing or imaging system, such as a standard 3D ultrasound (3DUS) system, can be used to produce images and/or image data of the catheter tip and the tissue target, and real-time image processing algorithms can be used to track the catheter-tissue relationship and control the motion of the guide-wire tip. The guide-wire tip can be translated in and out of the sheath to compensate for the cardiac motion, as well as other motion (e.g., respiration or patient motion) and to perform repairs.

The operation of these systems in accordance with the invention reveals a number of challenges that result from quickly translating a guide-wire inside a plastic sheath, particularly friction and backlash. These factors result in position hysteresis and significant tip trajectory errors. In accordance with one embodiment of the invention, the system can include compensation for fiction and backlash. The compensation based controller can use motion prediction which compensates for known friction forces and applies backlash position error to compensate for dead zone behavior to provide accurate motion. In one embodiment, the system can use feed-forward Coulomb friction compensation to compensate for friction forces and reduce tracking errors. In other embodiments, the system can use image processing of the 3-D ultrasound images to control the motion of the catheter and reduce tracking errors.

In alternative embodiments of the invention, the sensing or imaging system can include magnetic resonance imaging (MRI), X-ray computed tomography (CT), electromagnetic tracking systems, infrared imaging, m-mode ultrasound, 2D ultrasound, and a range of x-ray technologies including fluoroscopy. In addition, various combinations of these sensing and imaging systems can be used together to provide enhanced sensing and imaging to produce data and images that can be used to track the positions of the catheter tip and the target tissue.

In other embodiments of the invention, the catheter can provide the tip with more than one degree of freedom motion to allow for more complex movements of the catheter tip and provide multiple degree of freedom compensation to enable accurate tracking more complex tissue motion.

In other embodiments of the invention, the catheter can be controlled and actuated by one or more actuators (e.g., motors, linear actuators) located inside the catheter body and/or at the catheter tip. These actuators can be provided as an alternative to or in addition to actuators located at the base of the catheter.

In other embodiments of the invention, the catheter can include force sensors, for example, coupled to the guide-wire at the base of the catheter or mounted at the tip of the catheter, to enable the system to compensate for motion of the tissue or control the interaction of the device with the tissue. For example, a force sensor can be used to control the tip in order to maintain a predefined constant force or maintain a predefined varying force profile on the target tissue, while the tissue is in motion.

In accordance with implementations of the invention, one or more of the following capabilities may be provided.

It is one object of the invention to provide a catheter that is actuated and provides compensation for fast moving target tissue.

It is another object of the invention to provide a motion compensated catheter tool that uses 3-D imaging for guidance.

It is another object of the invention to provide a motion compensated catheter tool that uses 3-D imaging for closed-loop control of the motion.

It is another object of the invention to provide a motion compensated catheter having improved tip position accuracy.

It is another object of the invention to provide a motion compensated catheter having improved tip force accuracy.

It is another object of the invention to provide a motion compensated catheter that provides tissue tracking at speeds greater than manual motions.

It is another object of the invention to provide a system that can track fast moving tissue structures, such as fast moving in-cardiac structures.

It is another object of the invention to provide a system that can move at speeds of at least 210 mm/s with an acceleration of at least 3800 mm/s².

These and other capabilities of the invention, along with the invention itself, will be more fully understood after a review of the following figures, detailed description, and claims.

### BRIEF DESCRIPTION OF THE FIGURES

FIG. 1 is a diagrammatic of a catheter system according to the invention.

FIG. 2 is a diagrammatic view of a catheter system according to an embodiment of the invention.

FIG. 3 shows a flow chart of a process for providing motion compensation according to one embodiment of the invention.

FIG. 4 shows diagram of a catheter including a sheath and a guide-wire and illustrating the gap size, bending angle and the bending radius.

FIG. 5 shows a graph depicting the friction force versus velocity for a catheter system according to one embodiment of the invention.

FIG. 6 shows a graph depicting the desired sinusoidal tip trajectory, the actual tip trajectory with out compensation and the tip trajectory with compensation.

FIG. 7 shows a Position hysteresis curve for a sheath-guide-wire system according to an embodiment of the invention having a 3 mm backlash width following a sinusoidal trajectory.

FIG. 8 shows a diagram of guide-wire position in the sheath under tension and a diagram of guide-wire position in the sheath under compression.

FIG. 9 shows a graph of the backlash model error as function of friction.

FIG. 10 shows a graph that includes the mitral valve annulus trajectory (the desired trajectory), the tip trajectory, and the improved tip trajectory obtained using inverse compensation.

FIG. 11A shows human mitral valve annulus trajectory and FIG. 11B shows the corresponding force disturbances.

FIGS. 12A and 12B show a block diagram and an isometric diagram of a tip force sensor in accordance with one embodiment of the invention. FIGS. 12C and 12D show a diagrammatic view of a tip force sensing system according to an alternate embodiment of the invention. FIGS. 12E and 12F show a diagram of a tip force sensing system according to an alternate embodiment of the invention.

FIG. 13 shows a diagram of the system for modeling the catheter system according to one embodiment of the invention.

FIG. 14 shows a flow diagram of a force control system according to one embodiment of the invention.

FIG. 15 shows an ultrasound image showing the catheter and the target issue according to the invention.

FIG. 16 shows a graph that includes the trajectory of the target surface, the catheter base, and the catheter tip of a system according to the invention.

FIG. 17A shows a graph of the trajectory of the catheter tip and the mitral valve annulus found by manual segmentation according to one embodiment of the invention and FIG. 17B shows a graph of the catheter trajectory tracking error.

### DETAILED DESCRIPTION OF PREFERRED EMBODIMENTS

This present invention is directed to a catheter system that employs motion compensation techniques and optionally, other corrections, in order to minimize invasiveness in surgical procedures involving moving target areas, tissue and organs, such as a beating heart. In accordance with one embodiment of the invention, the system includes an actuator at the base of the catheter system that can drive a catheter guide-wire inside the flexible catheter sheath. For example, the sheath can be manually or automatically manually advanced through the vasculature into the heart. A standard 3-D ultrasound (3DUS) probe can be used to produce images of the catheter tip and the tissue target, and real-time image processing algorithms can be used to track the catheter-tissue relationship and control the motion of the catheter guide-wire tip. The guide-wire tip can be translated in and out of the sheath in correspondence with the cardiac motion to perform repairs. A control system can be used to track the target tissue motion and the guide-wire tip motion and to compensate for target tissue motion as well as other motion (e.g., respiration and patient motion). Additional motion compensation can be provided to compensate for mechanical factors such as friction and backlash to provide for more accurate tracking and positioning. Force control can be provided enable the tip to maintain a constant force or a predefined varying force profile on the target tissue, when the tip is placed in contact with the moving target tissue.

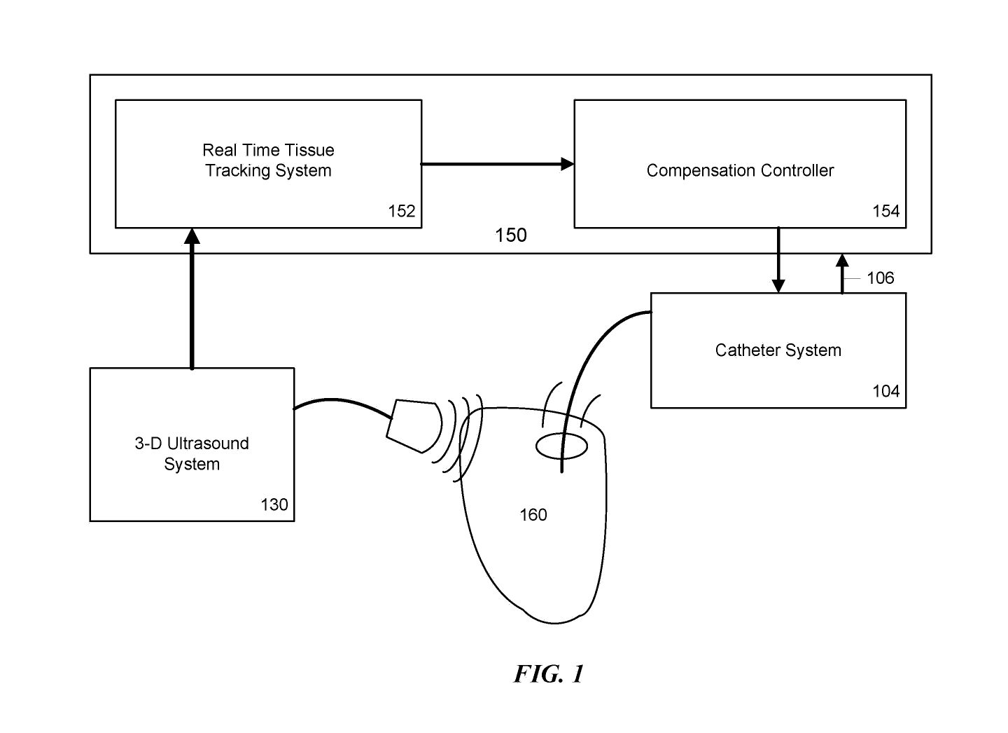

FIG. 1 shows a diagrammatic view of a system in accordance with one or more embodiments of the invention. The system can include a catheter system 104, including an actuator connected to the base end of the catheter guide-wire to move the catheter guide-wire within the flexible catheter sheath. The sheath can be manually advanced through the vasculature into the heart and positioned by a clinician. At the distal end of the catheter, inside the organ 160 (for example, a beating heart), the catheter system 104 controlled guide-wire tip translates in and out of the sheath under system 150 control to track the movement of the cardiac structures and perform repair. The system can include an imaging system 130, such as a 3 dimensional (3-D) ultrasound system, that produces image data, such as 3 dimensional image data (image volumes), of the organ 160 and the target area. A computer system includes software system 150 that uses the 3 dimensional image data to detect the motion of the guide-wire tip and the target tissue and control the movements of the catheter guide-wire to follow the movement of the target tissue. Software system 150 can include a real time tissue tracking system 152 that determines motion information of the target tissue within the organ 160 and provides the motion information to the compensation controller 154 which controls and coordinates the motion of the catheter guide-wire with the motion of the tissue. The compensation controller 154 can use sensor data 106 received from the catheter system 104 track the position and motion of the catheter guide-wire. The compensation controller 154 can also use sensor data 106 received from the catheter system 104 to control the force maintained by the tip on the target tissue.

In some embodiments of the invention, the tracking system and controller can be combined in a single system. For example, a compensation controller can include one or more processes, modules or elements for controlling the motion of the catheter guide-wire as well as signal processing modules or elements for processing sensor and imaging data using this data to control the motion of the motion of the catheter guide-wire. In one embodiment, the controller receives the sensor and image data and uses this data to determine and modify over time, the motion of the guide-wire.

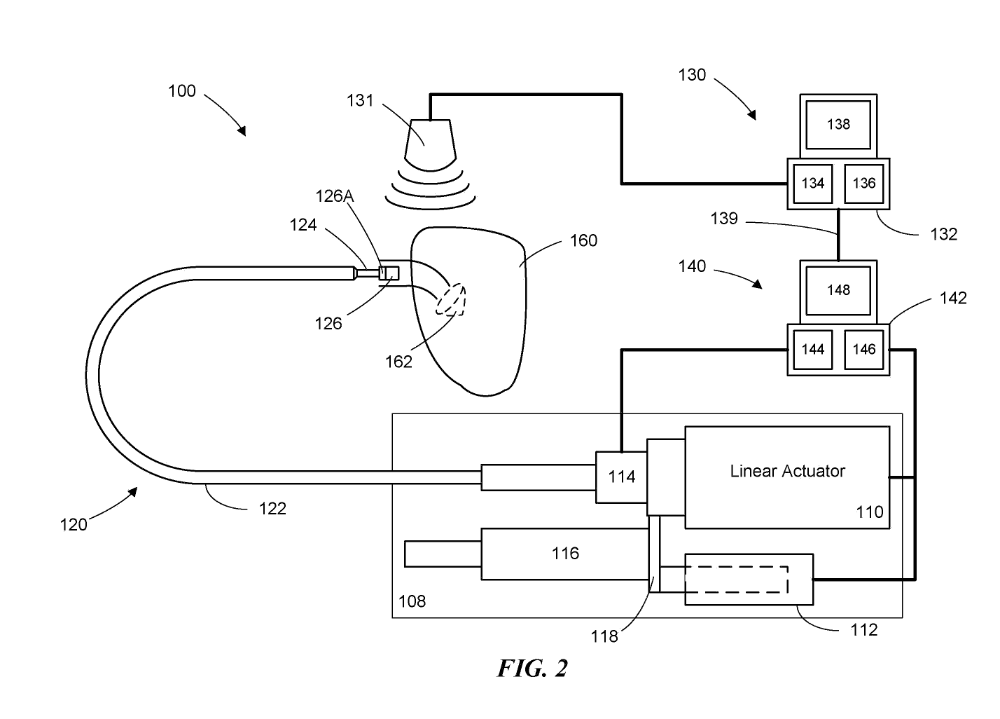

FIG. 2 shows a diagram of a system 100 according to one embodiment of the invention, a catheter 120 based system that can be used to repair a mitral heart valve while the heart continues to beat. The system 100 can include, a rigid base 108, a linear actuator 110 mounted to base 108, a potentiometer 112, mounted to base 108 and coupled to the linear actuator 110, a force sensor 114 coupled to the linear actuator and the catheter 120 and a linear slide 116, mounted to base 108 and connected to the linear actuator 110 and the potentiometer 112 by arm 118. The base of the catheter 120 can be coupled to the linear actuator 110 through the optional force sensor 114. The catheter 120 can include a sheath 122, a guide-wire 124 that extends from the base of the sheath 122 out through the remote end of the sheath 122 and includes an end effector 126, such as tacking device or stapling device, used to perform repairs on the mitral valve 162 of the beating heart 160. The end effector 126 can include a force sensor 126A interposed either at the tip or between the guide-wire 124 and the end effector 126. The guide-wire 124 can be fastened to the moving portion of the linear actuator 110 and the sheath 122, along with the stationary portion of the linear actuator 110 can be fastened to the rigid base 108, such that operation of the linear actuator 110 causes the guide-wire 124 to move relative to the sheath 122. The motion of the linear actuator 110 is linear, and the sheath 122 and the guide-wire 124 can be flexible allowing the tip 126 of the guide-wire 124 to move along axes that extend in any angle with respect to the axis of motion of the linear actuator 110, including axes that are perpendicular, as well as parallel, to the axis of motion of the linear actuator 110.

The system 100 can also include a computer system 140 that can include one or more central processing units (CPUs) 144 and associated memory 146 (including both volatile and non-volatile memory devices) for storing programs and data that control the linear actuator 110 and receive signals from the potentiometer sensor 112 and the force sensor 114 via cables (shown or wireless media, not shown). Computer system 140 can also include one or more displays 148 that can present information about the operation of the system 140 as well as images of the procedure to the operator and/or clinician. The system 100 can also include an imaging system 130, such as a 2 dimensional (2-D) or a 3 dimensional (3-D) ultrasound system which can include a control unit 132 connected by a cable to an image sensor 131 such as a 2-D or 3-D ultrasound probe, a camera or scanner. The control unit 132 of the imaging system 130 can include a computer system that has one or more CPUs 134 and associated memory 136 (including both volatile and non-volatile memory devices) for storing programs and data that control the operation of the imaging system 130. The imaging system 130 can also include one or more displays 138 that can present information about the operation of the system 130 as well as images of the procedure to the operator and/or clinician. The imaging system 130 and the computer system 140 can be connected by a cable 139 (shown, or a wireless connection, not shown) to allow data to be transferred between the imaging system 130 and the computer system 140. The imaging system 130 can be, for example, a SONOS 7500 system available from Philips Healthcare, Andover, Mass., USA. Alternatively, the imaging system can be magnetic resonance imaging (MRI), X-ray computed tomography (CT), electromagnetic tracking systems, infrared imaging, m-mode ultrasound, 2D ultrasound, Intracardiac Echocardiography (ICE) and a range of x-ray technologies including fluoroscopy. In some embodiments of the invention, these imaging systems can be used in 2-D or 3-D modalities, performing compensation based on 2-D or 3-D image data. In some embodiments of the invention, these imaging technologies can be used inside the body, for example, using a second catheter or mounting the imaging system to the guide-wire tip or a second guide-wire. In addition, various combinations of these sensing and imaging systems can be used together to provide enhanced sensing and imaging to produce data and images that can be used to track the positions of the catheter tip and the target tissue. Computer system 130 and computer system 140 can be Windows (from Microsoft, Redmond, Wash.), Linux, or Macintosh (from Apple, Inc. Cupertino, Calif.) based computer systems.

In operation, the operative end or tip 126 of the catheter can be manually or automatically inserted into the vasculature, such as femoral artery, and advanced into position adjacent the mitral valve in the heart. In some embodiments, the catheter can be steerable to control the orientation of the end of distal end of the catheter 126. The imaging system 130 can be used to guide the tip 126 of the catheter in position near the target tissue. Once in position, the imaging system 130 can be used to produce real time, 3 dimensional images (sets of pixel data or voxel data) of the moving tissue (for example, a mitral heart valve) and tip 126 of the catheter. This 3-D image data can be transferred to computer system 140 where real time image processing algorithms can be used to track the tissue movement and tip 126 of the catheter. These image processing algorithms are well known, see, for example, S. G. Yuen, S. B. Kesner, N. V. Vasilyev, P. J. Del Nido, and R. D. Howe, 3D Ultrasound-Guided Motion Compensation System for Beating Heart Mitral Valve Repair, Medical Image Computing and Computer-Assisted Intervention (MICCAI), New York, N.Y., Sep. 6-10, 2008 and P. M. Novotny, J. A. Stoll, P. E. Dupont and R. D. Howe, Realtime Visual Servoing of a Robot using Three-Dimensional Ultrasound, in Proc. IEEE Int. Conf. Robotics and Automation, 2007, pp. 2655-60, and P. M. Novotny, J. A. Stoll, N. V. Vasilyev, P. J. del Nido, P. E. Dupont, T. E. Zickler, and R. D. Howe. GPU based Real-time Instrument Tracking with Three-Dimensional Ultrasound, Medical Image Analysis, 11:458-464, 2007, all of which are hereby incorporated by reference in its entirety.

These image processing algorithms can include a real-time GPU based Radon and Hough transforms to determine the ray that corresponds to the axis of motion of the guide-wire and a K-means algorithm to identify the guide-wire tip and the tissue in a 2D image generated from the 3D image data. Tissue tracking can be accomplished using a “flashlight real-time tissue tracker” algorithm which includes using the Radon transform to find the axis of the guide-wire 124 and then distinguish between the guide-wire tip 126 and the target tissue 162 by analyzing the image volume data along the determined axis of the guide-wire 124. The K-means algorithm can be used to identify the clusters corresponding to the locations of the tip 126 and the target tissue 162. This can be difficult when the tip 126 and target tissue 162 are close together, so an active contour algorithm can be used to more clearly identify the target tissue 162 in the image and provide more robust real-time target tissue segmentation. This is described in S. G. Yuen, N. V. Vasilyev, P. J. del Nido, and R. D. Howe, Robotic Tissue Tracking for Beating Heart Mitral Valve Surgery, Medical image Analysis, 8 Jul., 2010, which is hereby incorporated by reference in its entirety. Using the tracking information about the tip 126 and tissue 162 movements, the computer system 140 can control the linear actuator 110 to coordinate the movement of the tip 126 with respect to the moving tissue.

The computer system 140 can use signals received from the potentiometer 112 to determine the position and direction of movement of the linear actuator 110 and the tip 126 of the catheter 120. The computer system 140 can use signals received from the force sensor 114 to determine the forces experienced by the base end of the guide-wire 124 and signals from force sensor 26A to determine the forces experienced by the tip 126 of the guide-wire 124. Using this signal information, the computer system 140, executing one or more programs stored in memory, can monitor forces applied by the tip 126 and control the linear actuator 110 to control the motion of the guide-wire 124 in order to maintain a constant force or provide a predefined force profile.

The computer system 140, executing one or more programs stored in memory, can control the movement of the linear actuator 110 in order to control the movement of the guide-wire 124 and the tip 126. A position sensor, such as potentiometer 122, can be used to determine the position and motion of the linear actuator and, when combined with the image data, can be used to control the movement of the tip 126 with respect to the tracked movements of the moving tissue. In operation, the tip 126 of the guide-wire 124 can be placed in contact with the moving tissue in order to perform a predetermined repair operation, such as tack or staple torn tissue or fasten a prosthetic device in place.

In accordance with one embodiment, the design parameters for the actuated catheter system 100 can be selected from the human mitral valve physiology dimensions. In this embodiment, the system's 100 principal functional requirements are that it has a single actuated linear degree of freedom with at least 20 mm of travel that can provide a maximum velocity and acceleration of at least 210 mm/s and 3800 mm/s², respectively. These values have been shown to be sufficient to compensate for the human mitral valve motion, however other design values can be selected depending on the specific physiology, for example, a larger animal such as an elephant or a whale, would require larger scale system parameters and a smaller animal such as mouse, would require smaller scale system parameters. The catheter components in the device should have the same dimensions and material properties as current clinical cardiac catheters, which can be selected from a range based on the patient requirements (e.g. ranging from a small infant to a large adult). Finally, the system should be able to apply a sufficient force to modify cardiac tissue, approximately 4 N.

For example, the embodiment shown in FIG. 2 can be composed of a linear voice coil actuator 110 with 50.8 mm of travel and a peak force of 26.7 N (NCC20-18-02-1X, H2W Technologies Inc, Valencia Calif.), a linear ball bearing slide 116 (BX3-3, Tusk Direct, Inc., Bethel Conn.), and a linear potentiometer position sensor 116 (A-MAC-B62, Midori America Corp, Fullerton Calif.). The catheter sheaths 122 can be 85 cm long sections of Teflon tubing, and the guide-wires 124 can be stainless steel close-wound springs. A geometric description of examples of various combinations of sheaths and guide-wires is provided in Table 1.

TABLE 1
EXPERIMENTAL CATHETER DIMENSIONS

| | Sheath Inner | Guide-wire | |
|---|---|---|---|
| Symbol | Diameter (Dₛₕ) | Diameter (D_{gw}) | Gap Size (G) |
| ∇ | 1.59 mm | 0.76 mm | 0.83 mm |
| [symbol] | 1.59 mm | 1.50 mm | 0.09 mm |
| ◯ | 2.38 mm | 1.50 mm | 0.88 mm |
| □ | 2.38 mm | 2.23 mm | 0.15 mm |

For illustrative purposes, the system 100 can include two modules: the base module, which includes the linear actuator 110, the slide 116, and the potentiometer 112, and the catheter module 120, which includes the catheter sheath 122 and guide-wire 124. The catheter sheath 122 can be flexed or steered into different configurations as required by the vascular geometry (bent, twisted, etc.) while the guide-wire 124 can be servoed by the base module. In addition, the system can include an imaging system module 130 and a control system module 140.

In one embodiment of the invention, a proportional-integral-derivative (PID) control system running at 1 kHz can be used to control the position of the linear actuator 110 at the base of the catheter. Custom C++ code can be used to control the system and make sensor measurements via a data acquisition card (DAQCard-6024E, National Instruments Corp., Austin Tex.) in computer system 140. Optionally, commands from the computer system 140 to the linear actuator 110 can be amplified by a bipolar voltage-to-current power supply (BOP 36-12M, Kepco Inc., Flushing N.Y.). The friction reaction forces between the guide-wire 124 and the sheath 122 can be measured with a miniature force sensor 114 (LCFD-1KG, Omega Engineering, Stamford Conn., range: 10 N, accuracy: +/−0.015 N) connected to a differential amplifier (AM502, Tektronix, Beaverton Oreg.).

In accordance with some embodiments of the invention, the compensation system can provide for high speed and high accuracy compensation that cannot be achieved by manually (human) implemented procedures. As a person having ordinary skill would appreciate, the speed and performance of the tracking system and the compensation controller can vary depending on the desired application, but the level of speed and performance can be selected based on the motion parameters of the target tissue. Thus, the performance characteristics of the tracking system and the compensation controller can be selected to provide the desired level of tracking performance (e.g., least tracking error in terms of frequency, position and/or lag) and the desired level of compensation performance (e.g., least compensation error in terms of position and/or lag). For example, it may be desirable to use a tracking system and a compensation system that operates at greater than multiple (e.g., 2×, 3×, . . . 10×) the maximum frequency of the target tissue motion or an important component of the target tissue motion to provide the desired level of compensation.

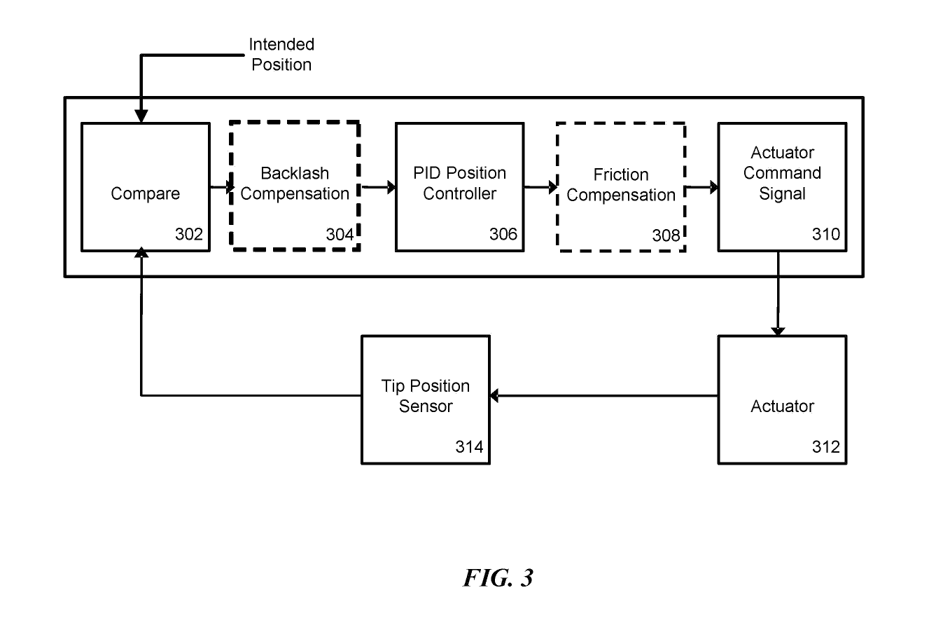

FIG. 3 shows a diagram of a component or software code module that can be used to control the position of the system by receiving sensor inputs, such as at the data acquisition card. At 302, the compare module compares the intended position of the catheter tip with tip position sensor data to determine the error signal in the tip position. The intended position of the catheter tip can be 1) a predefined point in space, 2) the current or anticipated location of the target tissue, or 3) a position relative to the current or anticipated location of the target tissue. At 304, optionally, a backlash compensation signal can be determined by a backlash compensation module and combined with the error signal. At 306, the PID position controller can use the error signal to determine a correction signal. At 308, optionally, a friction compensation module can determine a friction compensation signal and combine it with the correction signal. At 310, the correction signal can be used to produce an actuator command signal. The actuator command signal can be output to actuator 110 at 312. The actuator 110 moves in response to the actuator command signal causing the guide-wire and the catheter tip to move. At 314, the tip position sensor generates new tip position data which is input into the compare module and the process repeats.

In one embodiment, the catheter tip 126 position, for calibration purposes, can be measured with an ultra-low friction rotary potentiometer (CP-2UTX, Midori America Corp, Fullerton Calif.). The linear motion of the tip 126 can be converted into rotation of the potentiometer through a lightweight lever arm that connects the tip 126 of the catheter to the sensor. A long lever arm can used to ensure that the measurements are linear. In an alternative embodiment, including a clinical setting, the tip position can be measured with an electromagnetic tracker or ultrasound imaging.

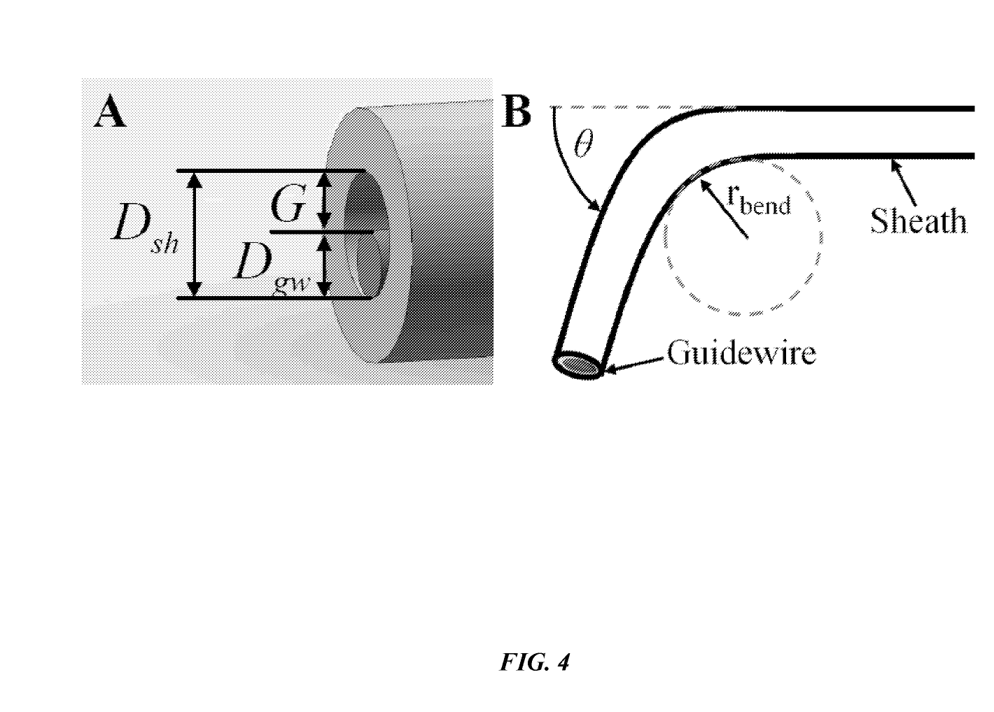

In accordance with one or more embodiments of the invention, the system 100 can compensate for the friction forces experienced by the guide-wire 124 and the backlash behavior exhibited by the guide-wire-sheath interaction. These two properties of the system degrade the trajectory tracking accuracy and response time of the actuated catheter end effector at the tip 126. A large number of factors can be considered in determining the friction and backlash properties of the catheter system. In accordance with one embodiment of the invention, the factors can include the gap size between the sheath and guide-wire and the bending configuration of the catheter, characterized by the bend radii and bend angles of the sheath. FIGS. 4A and 4B show the factors and the corresponding variables: where Dsh is the inside diameter of the sheath, Dgw is the outer diameter of the guide-wire, G is the gap, θ is the bend angle of the catheter sheath and r_{bend} is the bend radius of the catheter sheath.

In accordance with the invention, the friction experienced by the catheter system can be examined as a function of four different sheath-guide-wire gap sizes (Table 1 and FIG. 4A-4B), three bending angles (90°, 180°, and 360°), and two bend radii (25 and 50 mm). The friction can be calculated by commanding a series of constant velocities from the actuator in both the positive and negative directions. Force sensor readings during the constant velocity portion of the trajectory can be averaged and plotted against the velocities. The friction data was summarized for each configuration by taking the average of the determined friction values.

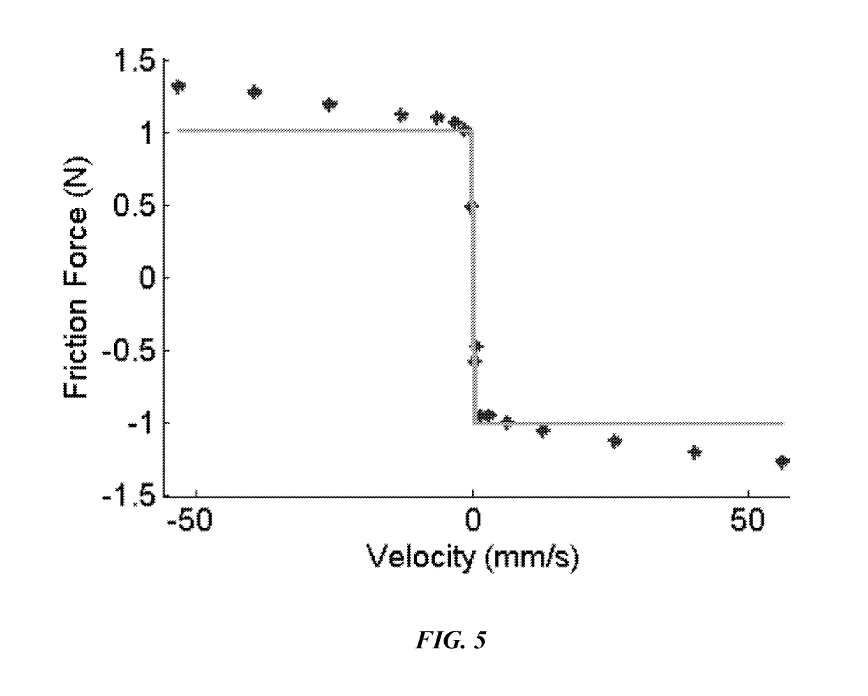

FIG. 5 presents a typical friction-velocity curve for systems in accordance with various embodiments of the invention. The observed behavior can be approximated as constant dynamic friction plus a component that varies linearly with velocity. For this case, the Coulomb term can be approximated as 1.0 N of friction, and the velocity dependent term as 0.006 N/(mm/s). Configurations with less than 0.05 N of friction were assumed to be frictionless because the friction was on the order of the sensor drift for this embodiment.

The system movement data can be analyzed using a three-way analysis of variance (ANOVA) to assess the operation of each specific system, as each can have its own performance characteristics based on the character of the components selected. The most significant result is that the gap size has the strongest influence on guide-wire friction (p<0.0001). The gap size, the interior space between the guide-wire and the sheath (FIG. 4A), directly affects the normal forces applied to the guide-wire by the sheath. The normal force is created by any sections of the sheath that might be pinch or kinked, locations where the guide-wire is constrained to conform to the inner wall of the bending sheath, and guide-wire or sheath discontinuities that cause the two components to come into contact. The small gap size amplifies these properties because smaller deformations in the catheter system cause the sheath and guide-wire to interact. Large gap sizes, on the other hand, allow more space for misalignments. Therefore, increasing the gap size decreases the friction experienced by the guide-wire.

The analysis also shows that bend angle has an effect on the friction forces (p=0.004). One reason for this trend is that bending causes the sheaths' cross sections to deform slightly. This deformation can pinch the guide-wire, thus increasing the applied normal forces. Also, the bending of the sheath forces the inner guide-wire to also bend in order to conform to the outer sheath. The reaction forces generated by the conforming guide-wire increase the normal force and therefore the friction on the guide-wire. The bending radii used in this study, which span the typical range for cardiac catheters, do not appear to have an impact on the friction measurements (p=0.64).

These results indicate that for certain conditions, only the gap size and catheter bending are needed to estimate the friction in the system. However, the large number of additional factors that impact the friction experienced by the guide-wire, including the sheath and guide-wire materials and dimensions, the catheter seals and connectors, and the external forces applied to the system, make developing a general model of the system friction challenging.

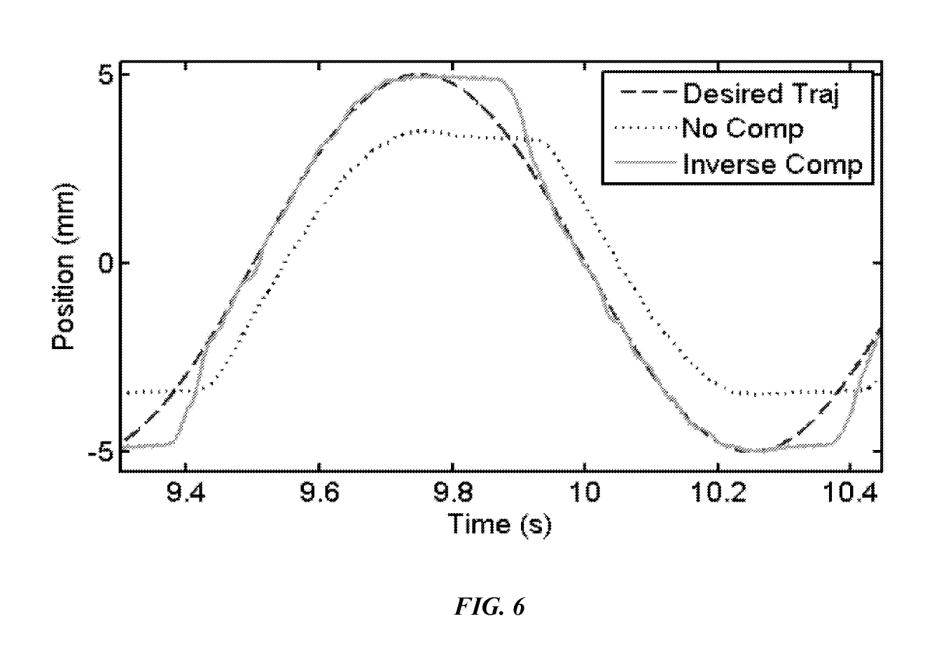

In accordance with the invention, the backlash properties of the sheath-guide-wire system can be investigated with the same variables (gap size, bend angle, bend radius) as for friction analysis above. The backlash can be examined by commanding the base of the catheter system to follow a 5 mm, 1 Hz sinusoidal trajectory (FIG. 6). This sample trajectory is a highly simplified version of a mitral valve annulus motion of a heart beating at 60 beats per minute (BPM). The hysteresis curve for the system plots the commanded versus measured tip position trajectory.

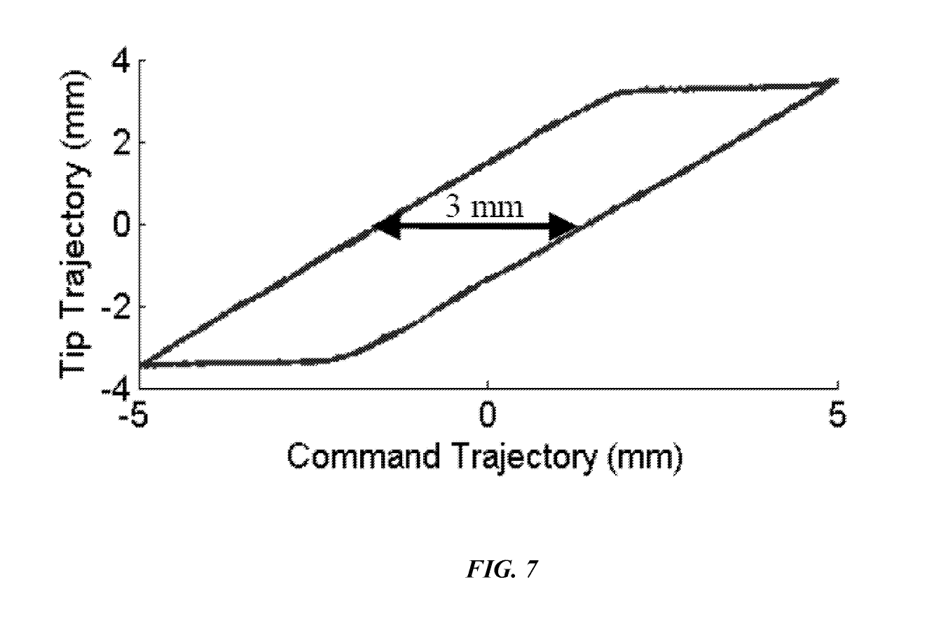

The amount of backlash can be quantified for each experiment by the width of the backlash hysteresis curve. For example, the hysteresis curve in FIG. 7 has a width of approximately 3 mm. The width of the hysteresis is the amount of displacement commanded at the base of the catheter that does not result in any movement at the tip.

The backlash data can be analyzed using a three-way ANOVA. In accordance with the invention, the bend angle has the clearest effect on backlash (p<0.0001) and the backlash width was found to be approximately proportional to the bend angle. In addition, the other parameter that was found to affect the backlash was the gap size (p<0.0001). While the gap size value did not proportionally relate to backlash, the data does suggest that the larger the gap size, the larger the possible amount of backlash. Bend radius was not found to have a significant effect on the backlash width (p=0.53).

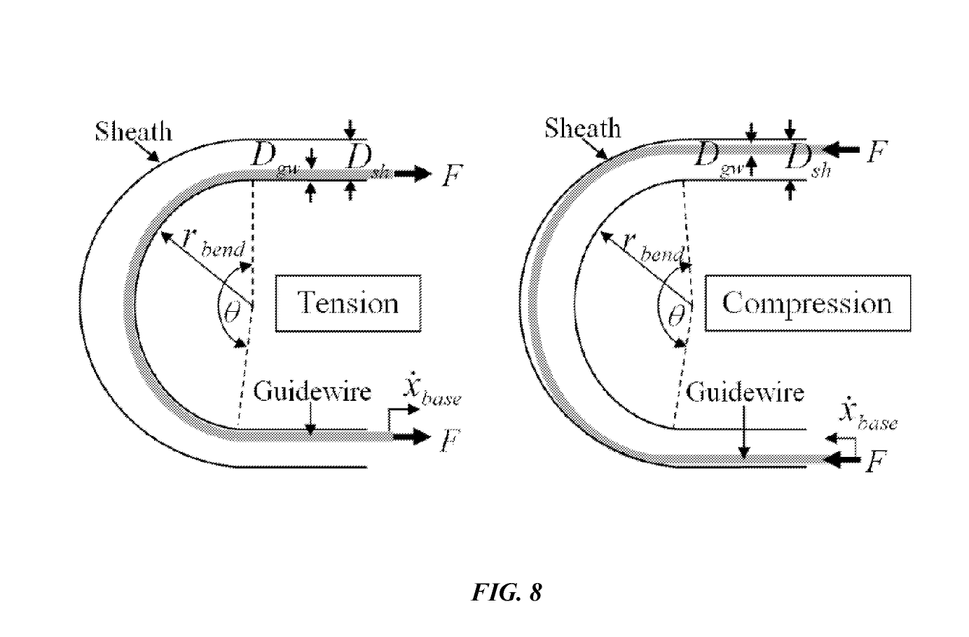

In accordance with the invention, a model can be used to explain the backlash width values in these results. The catheter guide-wires utilized in systems according to the invention are different from tendon transmission mechanisms because unlike tendons, the guide-wires are used both in tension and compression, which can result in buckling. Unlike backlash models that describe the effects of backlash on displacement and force transmission, our model, in accordance with the invention predicts the size of the backlash dead zone. If there is a bend in the catheter sheath, the guide-wire conforms to the bend inside of the sheath. Under tension, the guide-wire uses the inside of the curve as a bearing surface and slides along this inner surface of the sheath. When the guide-wire force changes directions to compression, the guide-wire is forced to switch positions and conform to the outside of the sheath, as shown in FIG. 8. As the force F switches from pulling the guide-wire in tension to pushing it in compression, the guide-wire tip does not initially move despite the translation of the base. In accordance with the invention, the backlash width is a function of the change in the length of guide-wire required to conform to the curvature inside the sheath. This change depends on the physical configuration and dimensions of the system embodying the invention. In accordance with one or more embodiments of the invention, the backlash width B can be predicted as the change in curve length

B=θ(r_{bend}+Dsh−½Dgw)−θ(r_{bend}+½Dgw) (1)
where θ is the bend angle of the sheath, r_{bend} is the bend radius of the sheath, Dsh is the inner diameter of the sheath, and Dgw is the diameter of the guide-wire. This equation can be simplified to a function of the bend angle and gap size

B=θ(Dsh−Dgw) (2)

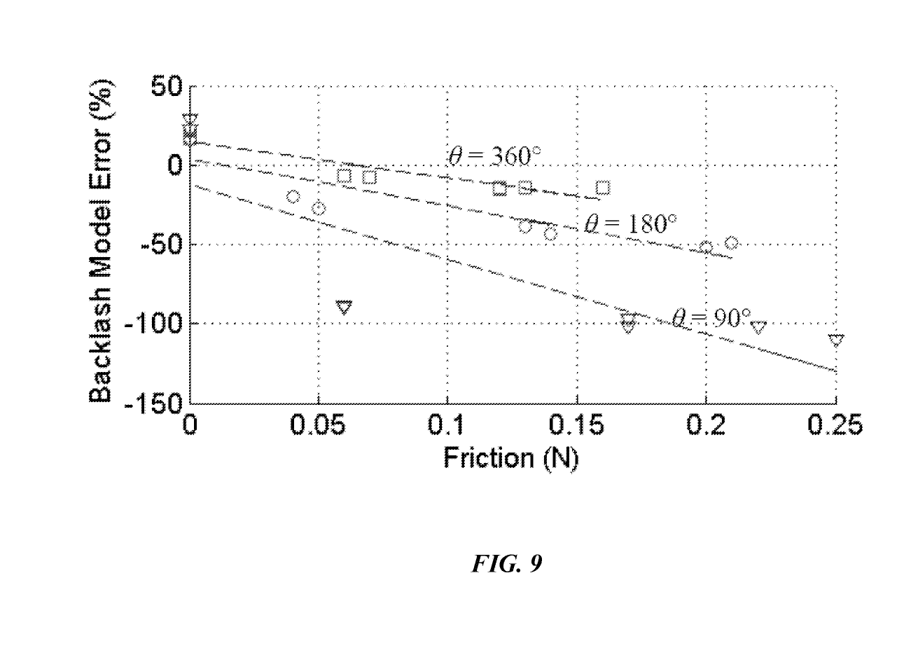

The backlash model of equation (2) is shown in FIG. 9. The model predicted values were plotted against the experimental data. The root mean square (RMS) error for the model was determined to be 0.4 mm and the coefficient of determination r² is 0.93.

These results show that the model accurately predicts the backlash width. The model slightly underestimates the backlash for lower backlash values and overestimates for larger values. This trend is probably caused by the level of friction experienced by the guide-wire. Systems with smaller gap sizes have greater friction, which causes the guide-wire to buckle in compression during operation and deforms the outer flexible sheath, thus increasing the backlash width. Systems with larger gaps experience decreased friction forces, which in turn reduce the forces that drive the guide-wire to conform to the inner wall of the sheath.

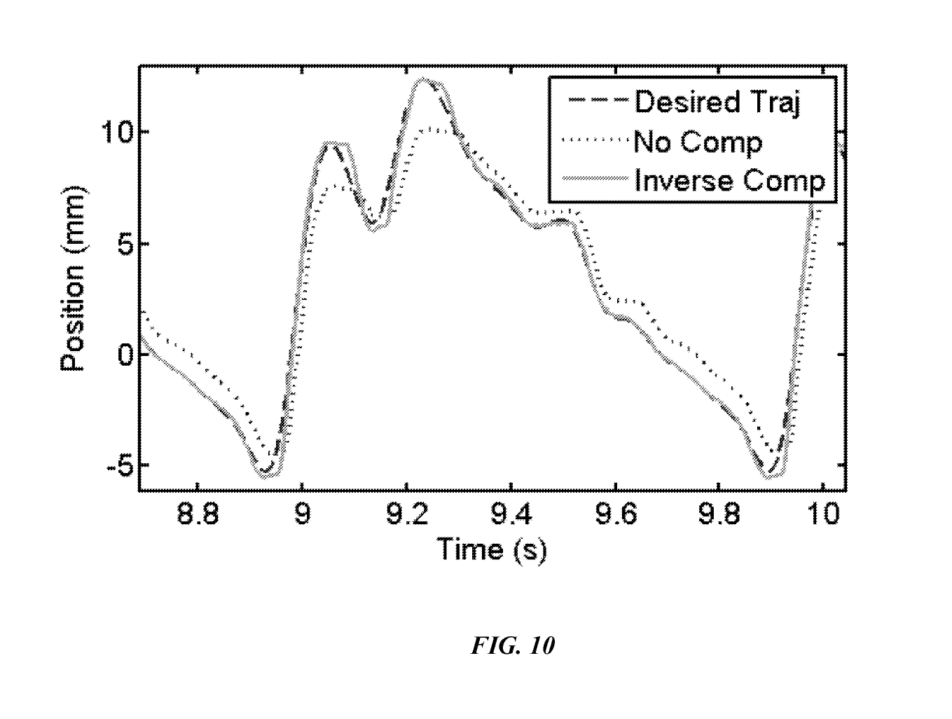

To evaluate the effects of guide-wire friction forces on the accuracy of the backlash prediction model, a 0.76 mm diameter guide-wire and a 1.59 mm inner diameter sheath were commanded to follow a 1 Hz trajectory in various bend angle configurations. The amount of friction experienced by the guide-wire was varied by applying a normal force to the sheath at the tip end of the guide-wire. The results, presented in FIG. 10, support the hypothesis that the amount of catheter friction affects the accuracy of the backlash model. The trends show that the model percent error decreases as the friction increases for each configuration. This understanding of how the amount of backlash is affected by friction resistance can be used to improve backlash compensation.

As demonstrated, the factors that affect catheter system trajectory tracking performance include friction and backlash. Knowledge of these factors can be used to improve system performance through both mechanical design and control system modifications. Friction in the catheter system can be reduced through material selection, material coatings, and lubrication. The backlash can be decreased by reducing the gap between the guide-wire and the sheath. However, reducing the gap can also increase the amount of friction experienced by the guide-wire. As a person having ordinary skill in the art will appreciate, this design tradeoff can be evaluated in order to improve system performance in view of design constraints of systems in accordance with one or more embodiments of the invention.

The system errors due to backlash and friction can also be reduced through improvements to the control system. For example, feed-forward Coulomb friction compensation can be used to reduce the friction force effects in the base module. However, friction compensation primarily improves the trajectory tracking of the base module. It is not able to reduce the main source of trajectory tracking error at the tip, the backlash behavior of the guide-wire. While the backlash is related to the friction resistance in the catheter, compensating for the friction at the base actuator does not reduce the backlash effects on the guide-wire.

In accordance with one or embodiments of the invention, the control system can reduce the backlash behavior by modifying the trajectory commanded at the base of the catheter. For example, the trajectory can be extended to ensure that the tip of the catheter overcomes the backlash dead zone and reaches the desired location. For example, one method of compensation for the backlash dead zone according to the invention is to solve for the inverse of the backlash. This method, known as inverse compensation, consists of commanding the system to follow a new trajectory created by adding the tracking error to the original trajectory. Limitations of this method include that it assumes the system can traverse the dead zone region instantaneously and that the system backlash width is constant and not velocity-dependent.

In accordance with the invention, the inverse compensation method can be used with the actuated catheter system 100. In one embodiment, a 0.8 mm diameter guide-wire and a 1.6 mm inner diameter sheath were constrained to a configuration with two 90° bends that simulated a realistic anatomical approach of passing the catheter from the inferior vena cava into the right atrium with a 50 mm bending radius, crossing the atrial septum, and then turning towards the mitral valve with a 25 mm bend radius. A rubber seal attached to the end of the sheath simulated a valve used to prevent the space between the sheath and guide-wire from filling with blood.

In one embodiment, inverse compensation was applied to a 1 Hz sinusoidal trajectory. Initially, without compensation, the tip position trajectory tracking mean absolute error (MAE) for the sinusoidal trajectory was 1.28 mm. The inverse compensation trajectory improved the tip position trajectory tracking by 80%, to a MAE error of 0.26 mm. FIG. 10 shows the desired sinusoidal trajectory, the original catheter tip trajectory, and the improved tip trajectory using inverse compensation. The main reason the inverse trajectory does not perfectly remove all of the backlash behavior is because the guide-wire does not move instantaneously through the dead zone region (e.g. near 9.8 sec in FIG. 10).

In one embodiment of the invention, the catheter system can be used to perform repairs inside the heart while it continues to beat. For example, the system can track a specific cardiac structure, the mitral valve annulus, the outer rim of the heart valve that sits between the left atrium and ventricle. Modification of this structure (i.e. mitral annuloplasty) is a common aspect of mitral repair surgery. The inverse compensation method can be applied to a typical mitral valve annulus trajectory taken from human ultrasound data as shown in FIG. 10. Without inverse compensation, the catheter tip failed to track the extremes of the mitral valve trajectory. However, the tip trajectory tracking greatly improved when the inverse compensation trajectory was applied to the system. The inverse method decreased the MAE error from 1.19 mm to 0.24 mm, an improvement of almost 80%.

While this compensation method can improve the catheter tip tracking, it requires the system to first follow the commanded trajectory inaccurately and then calculate how to alter the trajectory to improve tracking. This approach may be impractical for the real time control of some systems, because it assumes that the environmental conditions are constant during operation. In accordance with other embodiments of the invention, methods that would improve trajectory tracking in vivo include an adaptive compensator that updates a model of the system backlash based on the tracking performance or a repetitive control system that takes advantage of the periodicity of the cardiac motion. Another method involves providing close-looped control for the catheter tip position.

In accordance with one embodiment of the invention, the catheter motion control system according to one embodiment of the invention can be integrated with the (ultrasound) imaging based servoing system described herein and the combined system was evaluated in a water tank in vivo simulator. In accordance with one or more embodiments of the invention, the system can control a catheter to enable it to follow the motion of internal cardiac structures using real-time sensing of both the catheter tip and tissue target positions. Any real time volumetric imaging technique, such as 3D ultrasound can be used.

In accordance with one embodiment of the invention, the catheter motion control system according to the invention can include other imaging systems as an alternative to or in addition to the ultrasound imaging system described herein. For example, the control system can include an ultrasound system attached to a second catheter that is inserted via an alternate path through the vasculature or an opening created for the specific purpose of imaging the area to be treated. In an alternate embodiment, the ultrasound system can be a miniature system, built or mounted to the tip of the catheter or attached to the sheath of the catheter. Other imaging systems, such as MRI, CT, infrared and fluoroscopy (including biplane and single plane) can be used as an alternative to or in addition to the volumetric ultrasound system described herein. These systems, by themselves, can be used to detect tissue position and for tracking the motion of the tissue to be treated. These systems can be used in conjunction with volumetric imaging systems to detect tissue more precisely and for tracking tissue motion at higher speeds.

In an alternative embodiment of the invention, the catheter can include an extension that is adapted to attach itself to the tissue for support or bracing. For example, for a cardiac catheter, one or more additional guide-wires can be used to grab or brace against the septum or other tissue to support the catheter in position.

In alternative embodiments of the invention, the system can be used as a delivery system to deliver, implant, place or fasten a device such as a stent or other support device. In one embodiment, the present invention can be used to implant a cardiac stent.

Valve motion can be simulated using a cam follower mechanism that replicated the dominant 1-D motion component of the human mitral valve. The cam mechanism simulated a heart rate of 60 beats per minute. The moving target attached to the cam mechanism was extended into a water tank, to permit imaging by a SONOS 7500 3DUS machine (from Philips Healthcare, Andover, Mass., USA). A potentiometer can be used to measure target position for assessment of system accuracy.

In the (ultrasound) imaging based servoing system, 3D image volumes can be streamed via an Ethernet connection 139 to an image processing computer (FIG. 2). In accordance with other embodiments of the invention, other types of wired connections, such as USB and Firewire and wireless connections such as, WiFi, Bluetooth and Zigbee can be used. A GPU-based Radon transform algorithm can be used to find the catheter axis in real-time. The target tissue can be located by projecting the catheter axis forward through the image volume until tissue is encountered. This method allows the clinician to designate the target to be tracked by simply pointing at it with the catheter. To compensate for the 50-100 ms delay in image acquisition and processing, an extended Kalman filter can be used to estimate the current tissue location based on a Fourier decomposition of the cardiac cycle. In vivo experiments using this servoing system show that a rigid instrument system according to the invention is capable of accurate tracking within the heart, with an RMS error of 1.0 mm or less.

The intrinsic time delays in some imaging systems can make direct visual servoing of the catheter device potentially dangerous. The delays are estimated to be as much as 70 ms in the acquisition, transmission, and computation times—sufficient time for the mitral valve annulus to traverse the majority of its path at end systole. Left uncompensated, these delays could lead to collisions between the instrument and annulus that could result in tissue damage. To avoid this outcome, we exploit the near periodicity of the mitral valve trajectory to predict its path and hence compensate for time delay.

In accordance with the invention, one embodiment uses an extended Kalman filter (EKF) with an explicit quasiperiodic model, which is effective for 3DUS-guided mitral valve motion compensation. To model quasiperiodic heart motion, we consider the following m-order time-varying Fourier series with an offset

$$y ( t ) = c ( t ) + {\sum }_{i = 1}^{m} {r}_{i} ( t ) sin {θ}_{i} ( t ) \qquad ( 3 )$$

where y(t) is the target position in ultrasound coordinates, c(t) is the offset, rᵢ(t) are the harmonic amplitudes, and θᵢ(t)\hat{≡}i∫₀^{t}ω(τ)dτ+φᵢ(t), with heart rate ω(t) and harmonic phases φᵢ(t). In one embodiment, this parameterization was shown to be a robust model for mitral annulus tracking with m=8 harmonics.

Defining the state vector x(t)\hat{≡}[c(t), rᵢ(t), ω(t), θᵢ(t)]^{T}, iε(1, . . . , m) and assuming that c(t), rᵢ(t), ω(t), and θᵢ(t) evolve through a random walk, the state space model for this system is

$$x ( t + Δ t ) = F ( Δ t ) x ( t ) + μ ( t ) \\ z ( t ) = h ( x ( t ) ) + v ( t ) , \\ F ( Δ t ) = [ \begin{matrix}{I}_{m + 1} & & & & & 0 \\ & 1 & & & & \\ & Δ t & 1 & & & \\ 0 & 2 Δ t & 0 & 1 & & \\ & ⋮ & & & ⋱ & \\ & m Δ t & & & & 1\end{matrix} ] \qquad ( 4 )$$

where h(x(t))\hat{≡}y(t) from (1), v(t)˜N(0,σ_{R}²) is zero mean Gaussian noise, and μ(t)≈N(0,Q) is the random step of the states assumed to be drawn from a zero mean multivariate normal distribution with covariance matrix Q.

Prediction with this model requires estimation of x(t); a nonlinear estimation problem owing to the measurement function, h(x(t)). We employ the EKF, a nonlinear filtering method that approximates the Kalman filter through linearization about the state estimate \hat{x}(t−Δt|t−Δt). Other Kalman filters can be selected for use in the system according to the desired motion estimation and modeling of the catheter system and motion of the organ to be treated. Additional Kalman filters are described in P. Parker and B. Anderson, Frequency tracking of nonsinusoidal periodic signals in noise, Signal Process., vol. 20, pp. 127-152, (1990), which is hereby incorporated by reference. More information can be found in S. Yuen, P. Novotny, R. Howe, Quasiperiodic predictive filtering for robot-assisted beating heart surgery. In: Proc. IEEE ICRA, Pasadena USA (May 2008), which is hereby incorporated by reference in its entirety.

In accordance with one embodiment of the invention, the controller can be programmed to autonomously maintain prescribed forces of the tip against the target tissue despite its fast motion. It is desirable that in some applications, for example, beating heart surgery, the system must be damped and stable to ensure that it will not overshoot or oscillate in response to changes in the desired force trajectory or sudden target motions. Furthermore, it is desirable that the system have sufficient bandwidth to reject the disturbance caused by heart motion.

The inventors have determined that the vibrational modes in surgical instruments prevent the high gain settings required for a force regulator to obtain both damping and good heart motion rejection.

In accordance with one embodiment of the invention, the controller can incorporate feed-forward target motion information in order to provide safe and accurate force tracking at low bandwidth. This embodiment can include a force tracking system that functions in low bandwidth configurations by using feed-forward heart motion information derived from the imaging system to augment the controller. The system according to the invention can be used for beating heart mitral valve annuloplasty. The system can use a one degree of freedom actuated device that can follow the rapid, nearly uni-axial motion of the mitral valve annulus.

In accordance with the invention, the catheter system can be is modeled as a mass m and damper b subjected to a commanded actuator force fₐ and environment contact force fₑ. The damper b captures the effects of friction in the device, friction at the insertion point to the heart, and fluid motion. Approximating the environment as a spring of stiffness kₑ yields the system dynamics

m\ddot{x}+\dot{x}=fₐ−kₑ(x−xₑ) (5)
where x is the instrument tip position and xₑ is the desired tissue target position (i.e., its position if it were not deformed by contact). The model in equation (5) assumes rigid contact between the instrument and compliant target. For simplicity, we neglect force sensor compliance in the model because it is significantly stiffer than the tissue environment.

Now consider a standard force regulator control law:

fₐ=f_{d}+k_{f}(f_{d}−fₑ)−Kᵥ\dot{x} (6)
where K_{f} and Kᵥ are controller gains and f_{d} is the desired force. Combining (5) and (6) and applying the Laplace transform gives the closed-loop contact force relationship

Fₑ(s)=T(s)F_{d}(s)+Z(s)Xₑ₍ₛ₎ (7)
where the force tracking transfer function T(s), impedance transfer function Z(s), and the closed-loop characteristic C(s) are

$$\begin{matrix}T ( s ) \hat{≡} \frac{{F}_{e} ( s )}{{F}_{d} ( s )} = \frac{\frac{{k}_{e}}{m} ( 1 + {K}_{f} )}{C ( s )} & ( 8 ) \\ Z ( s ) \hat{≡} \frac{{F}_{e} ( s )}{{X}_{e} ( s )} = \frac{{k}_{e} s ( s + \frac{{K}_{v} + b}{m} )}{C ( s )} & ( 9 ) \\ C ( s ) \hat{≡} {s}^{2} + \frac{{K}_{v} + b}{m} s + \frac{{k}_{e}}{m} ( 1 + {K}_{f} ) & ( 10 )\end{matrix}$$

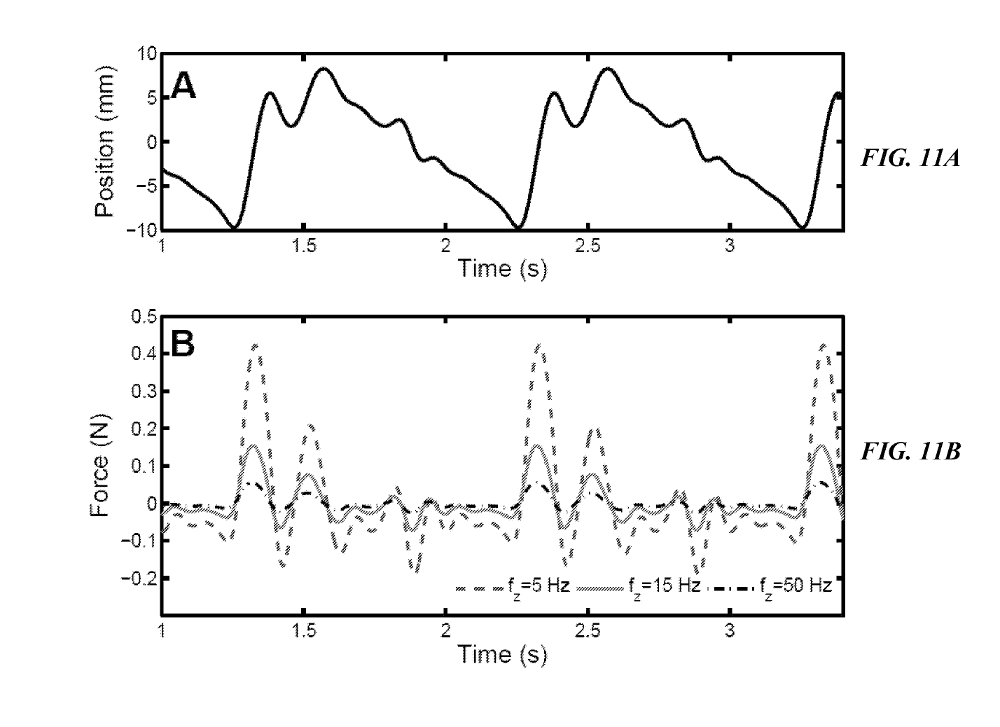

Equation (7) makes explicit that target motion xₑ is a disturbance that perturbs fₑ from f_{d}. Controller gains K_{f} and Kᵥ are chosen to ensure system stability, sufficient damping, and good rejection of xₑ. The last is achieved by designing Z(s) to have small magnitude in the bandwidth of Xₑ(s). For the mitral valve annulus, which is essentially band-limited to approximately 15 Hz, this is equivalent to setting the impedance corner frequency f_{z} greater than or equal to 15 Hz. FIG. 11A depicts typical mitral valve annulus motion and FIG. 11B shows its effect on the contact force for various f_{z} based on simulations of equations (7)-(9) with f_{d}=0. Parameter values of m=0.27 kg, b=18.0 Ns/m, and kₑ=133 N/m are assumed based on system identification of the device and preliminary estimates of the mitral valve annulus stiffness. As we will show shortly, obtaining a large impedance corner frequency f_{z} is synonymous with increasing the natural frequency fn of the closed-loop system, which is also equivalent to increasing K_{f}.

The natural frequency fₙ and damping ζ of the system can be expressed as

$$\begin{matrix}{f}_{n} = \frac{1}{2 π} \sqrt{\frac{{k}_{e}}{m} ( 1 + {K}_{f} )} & ( 11 ) \\ ς = \frac{{K}_{v} + b}{4 π {mf}_{n}} & ( 12 )\end{matrix}$$

To avoid potentially dangerous overshoot, we set the system to be critically damped (ζ=1.0). Manipulating equations (9), (11), and (12), it can be shown that the natural frequency fn of a critically damped system is a function of the impedance corner frequency f_{z} by

$${f}_{n} = {( \frac{\sqrt{208} - 14}{6} )}^{- \frac{1}{2}} {f}_{z} \approx 3.7698 {f}_{z} \qquad ( 13 )$$

Hence, while a position regulator can follow a trajectory band-limited to 15 Hz with about the same closed-loop natural frequency, a force regulator must have a natural frequency of approximately 57 Hz to follow the same motion. This indicates that the force regulator inherently requires high bandwidth to compensate for target motion. From equations (11)-(13) we calculate gain settings of K_{f}=255.2 and Kᵥ=173.8 to set our system to be critically damped with f_{z}=15 Hz, assuming that the rigid body model is appropriate at such high gains. It is clear that there is a trade-off between the two performance criteria: increasing K_{f} increases the corner frequency but decreases damping; the opposite is true for K. Because of this trade-off, achieving suitable disturbance rejection (f_{z}≧15 Hz) while maintaining damping (ζ≧1) requires large gains.

Consider the control law

fₐ=f_{d}+K_{f}(f_{d}−fₑ)+Kᵥ({dot over (\hat{x}ₑ+m{umlaut over (\hat{x}ₑ (14)
which is equation (6) augmented with feed-forward estimates of the target velocity {dot over (\hat{x}ₑ and acceleration {umlaut over (\hat{x}ₑ. The contact force relationship in (7) becomes

Fₑ(s)=T(s)F_{d}(s)−Z(s)(Xₑ(s)−\hat{X}ₑ(s)) (7a)
where T(s) and Z(s) are defined as before in (8) and (9), respectively. Observe that the use of feed-forward terms {dot over (\hat{x}ₑ and {umlaut over (\hat{x}ₑ enable the cancellation of the motion disturbance xₑ without the need to greatly increase the natural frequency of the system. The controller can then be designed with a low closed-loop natural frequency to avoid the effects of vibration and other high order dynamics that lead to reduced damping and instability. The feed-forward bandwidth can be set equal to the bandwidth of the heart motion disturbance, which is lower than the resonance frequency of the robot.

In accordance with one embodiment of the invention, a catheter system controller can be configured with the system parameters ζ=1.05 and f₀=8 Hz based on determined system parameters of m=0.27 kg, b=18.0 Ns/m, and preliminary estimates of mitral annulus stiffness kₑ=133.0 N/m. Using manual operation, contact with annulus yielded force standard deviations of 0.48±0.06 N (mean±std error). Using force control, the force deviations were reduced to 0.22±0.01 N. Using feed-forward force control, the force deviations were reduced further to 0.11±0.02 N.

In accordance with one embodiment of the invention, the surgical application can be performed on the mitral valve annulus inside of the beating heart using any real-time imaging technology that can image tissue through blood to allow for guidance. One such imaging technology is 3D ultrasound as it meets these criteria while providing volumetric information. In this embodiment, to obtain the motion terms used in the feed-forward controller, the position of the tissue in the ultrasound volume is determined using the real-time target tissue segmentation algorithm described herein. The algorithm takes advantage of the high spatial coherence of the instrument, which can appear as a bright and straight object in the volume, to designate the tissue target. The nearly uni-axial motion of the mitral valve annulus can be modeled as a time-varying Fourier series with an offset and truncated to m harmonics

$${x}_{e} ( t ) = c ( t ) + {\sum }_{i = 1}^{m} {r}_{i} ( t ) sin {θ}_{i} ( t ) \qquad ( 15 )$$

where c(t) is the offset, rᵢ(t) are the harmonic amplitudes, and θᵢ(t)\hat{≡}i∫₀^{t}ω(τ)dτ+φᵢ(t), with heart rate ω(t) and harmonic phases φᵢ(t). Prior to contact, measurements from the tissue tracker can be used to train an extended Kalman filter to provide estimates of the model parameters c(t), rᵢ(t), ω(t), and θᵢ(t). These parameters can be used to generate smooth feed forward velocity and acceleration terms for the force controller of equation (14) using the derivatives of equation (15). After contact, filter updates are stopped because the device interacts with the tissue, causing subsequent position measurements to no longer be representative of the feed-forward (i.e. desired) tissue motion.

In accordance with one embodiment of the present invention, the catheter applies a constant force on the moving cardiac tissue while performing a repair task on the beating heart, such as inserting surgical anchors for a mitral valve annuloplasty procedure. The force control capabilities can provide compensation for the high friction and dead zone backlash characteristics of the catheter system as well as the fast motion of the cardiac structures being operated on. It can be desirable for the control system to enable the catheter tip to apply a constant force on the moving target tissue.

In accordance with one embodiment of the invention, the catheter system can be designed to interact with the outer annulus of the mitral valve, located between the left atrium and ventricle. The system design parameters can be selected based on the application, for example, from human mitral valve physiology values. The principal functional requirements include a single actuated linear degree of freedom with at least 20 mm of travel that can provide a maximum velocity and acceleration of at least 210 mm/s and 3800 mm/s², respectively. These values have been shown to be sufficient to compensate for the human mitral annulus motion. The system can also be able to apply a sufficient force to modify cardiac tissue, approximately 4 N.

The complete system can be composed of three main modules: 1) the drive system that actuates the catheter, 2) the catheter module that is inserted through the vasculature into the heart, and 3) an imaging and control system such as a 3D ultrasound visual servoing system that tracks the tissue and commands the catheter to follow the motion (FIGS. 1 & 2). The drive system, shown in FIG. 2, includes a linear voice coil actuator with 50.8 mm of travel and a peak force of 26.7 N (NCC20-18-02-1X, H2W Technologies Inc, Valencia Calif., USA), a linear ball bearing slide (BX3-3, Tusk Direct, Inc., Bethel Conn., USA), and a linear potentiometer position sensor (CLP13, P3 America Inc., San Diego, Calif., USA). The catheter module (FIG. 3) is composed of a 70 cm long nylon sheath with a 2.70 mm inner diameter and an uncoated stainless steel coil guide-wire with a 2.39 mm outer diameter. The catheter sheath can be flexed as required by the vascular geometry (bent, twisted, etc.) while the guide-wire is servoed inside the sheath by the drive system.

Custom C++ code can be used to control the system and make measurements via a data acquisition card at 1 kHz. Commands to the linear actuator can be amplified by a linear current amplifier (AMPAQ, Quanser Inc., Markham, Ontario, Canada). For clinical implementation, the target position can be tracked using a 3D ultrasound imaging system that streams 3D images of the interior of the heart at video frame rates, for example 28-30 fps.

The catheter tip force sensor can be created with the design specifications of less than 5.5 mm outer diameter, less than 1 mm deflection under maximum load of 10 N, RMS errors less than 0.2 N, and good immunity to lateral forces. Preferably, the package can also accommodate an electromagnetic (EM) tracking sensor and allow for good integration with the catheter system.

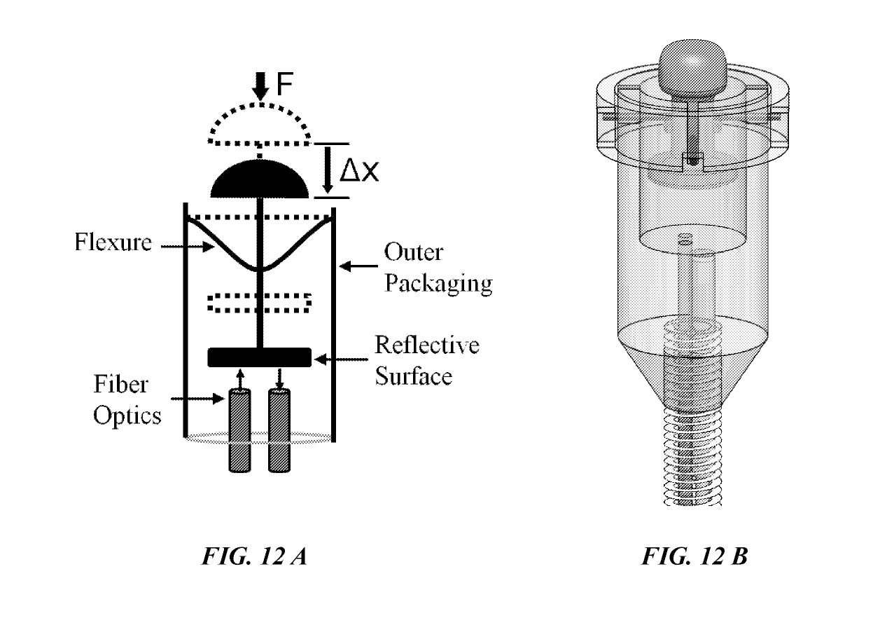

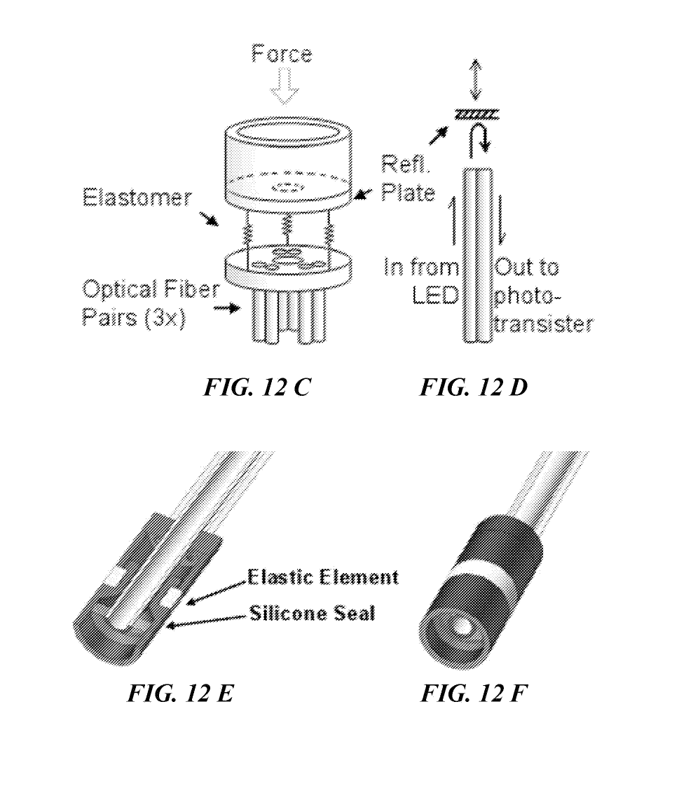

FIGS. 12A and 12B show one embodiment of a catheter force sensor design according to the invention. The force sensor can be coupled to then end of the catheter, wherein a first and a second fiber optic cable extend through the hollow catheter guide-wire into the sensor. The sensor can include a contact tip that makes contact with the target tissue. Preferably, the contact tip can be rigidly coupled, such as by a shaft, to a reflective surface. The contact tip can be supported by flexures at the distal end of the outer packaging of the tip and light emitted from the first optical fiber is reflected off the reflective surface and transmitted via the second optical fiber to an optical intensity sensor, such as a phototransistor circuit. In operation, the tip makes contact with an object or the target tissue and is displaced against the force of the flexures. The optical sensor can be used to detect displacement of the reflective surface relative to the ends of the fiber optic cables. The change in light intensity is indicated as change in voltage by the sensor, from which the displacement (distance) and force (as function of the displacement of the flexures) can be determined. The contact tip can include a central hole to allow a surgical implement, such as a 14 gauge needle for deployment of surgical anchors.

The structure of the force sensor can be fabricated using 3D printing (Objet Connex500 3D Printer, Objet Geometries Ltd, Billerica, Mass., USA). Nitinol wire flexures (0.25 mm diameter) can be used to provide sensor movement and arranged in a perpendicular configuration as shown in FIG. 12B. The sensor operates by converting the displacement of the flexures into a force value using a nonlinear calibration. One advantage of the fiber optic sensor is that it does not require current-carrying wires within the heart.

FIGS. 12C, 12D, 12E and 12F show an alternative embodiment of the force sensor design. In this embodiment, as shown in FIGS. 12C and 12D, the force sensor can be supported by an elastomer and can include 3 pair of optical fibers equally spaced around the tip. The tip can a reflector plate that reflects light transmitted from an LED through one of the optical fibers in to the return optical fiber to a photodiode. The force can be detected as a function of the change in reflected light intensity of one or more of the optical fiber pairs. As shown in FIGS. 12E and 12F, the contact tip can include a central hole to allow a surgical implement, such as a 14 gauge needle for deployment of surgical anchors.

An electromagnetic (EM) tracker can be integrated into the catheter tip to provide position information (3D Guidance trakSTAR, Model 90 sensor, 0.9 mm diameter, Ascension Technology Corp. Burlington Vt., USA). The EM tracker can have a sub-millimeter spatial resolution but high (>20 ms) latency that limits its use for accurate and stable close-looped control.

In accordance with one embodiment of the invention, the controller applies a desired force on a fast moving target with the catheter system. A standard error-based force control law is

Fₐ=F_{d}+K_{f}(F_{d}−Fₑ)−Kᵥ\dot{x} (16)
where Fₐ is the actuator force, F_{d} is the desired force, Fₑ is the force applied to the environment, K_{f} and Kᵥ are controller gains, and \dot{x} is the guide-wire tip velocity. However, this control approach has applicability for the motion compensated catheter system because of the inherent limitations, including backlash and friction in the catheter transmission system. These limitations prevent the force regulator in (16) from correctly responding to the force tracking error in a stable manner because the internal dynamics of the catheter obstruct the controller action from being accurately transmitted to the catheter tip. For example, as the target changes directions, the backlash in the catheter prevents the forces applied by the catheter from immediately changing. Therefore, there is a larger force tracking error that produces an even larger response from the force regulator. This can result in instability or the system entering a limit cycle.

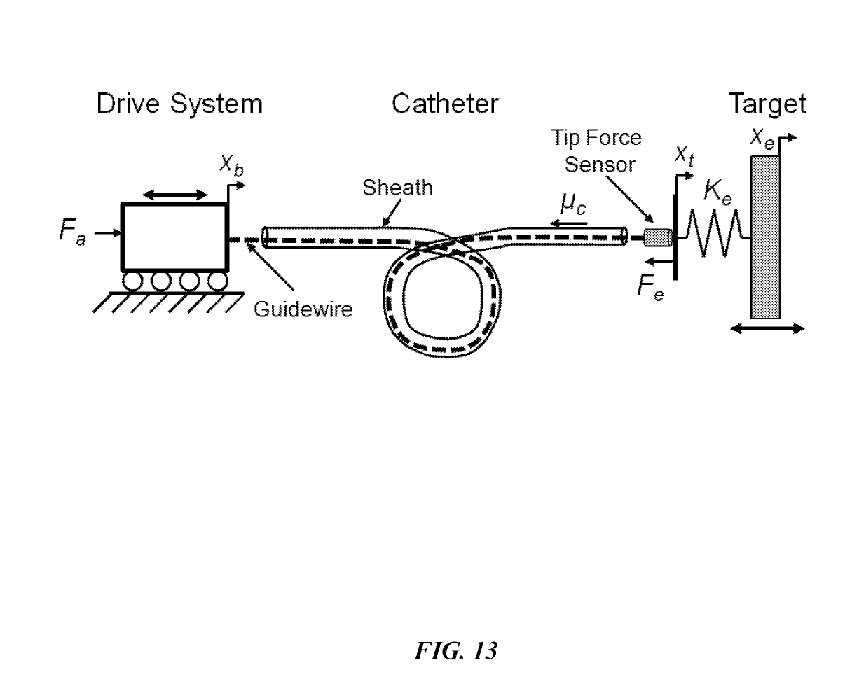

In accordance with one embodiment of the invention, the control system can use a force error term to modulate the commanded position trajectory of the catheter. FIG. 13 shows a diagram of the catheter system experimental setup and the system model variables.

In accordance with one embodiment of the invention, the drive system is commanded to follow a desired position, x_{d} that is the sum of the position of the moving target, xₑ and the position offset required to maintain the desired force, x_{f}

x_{d}=xₑ+x_{f} (17)

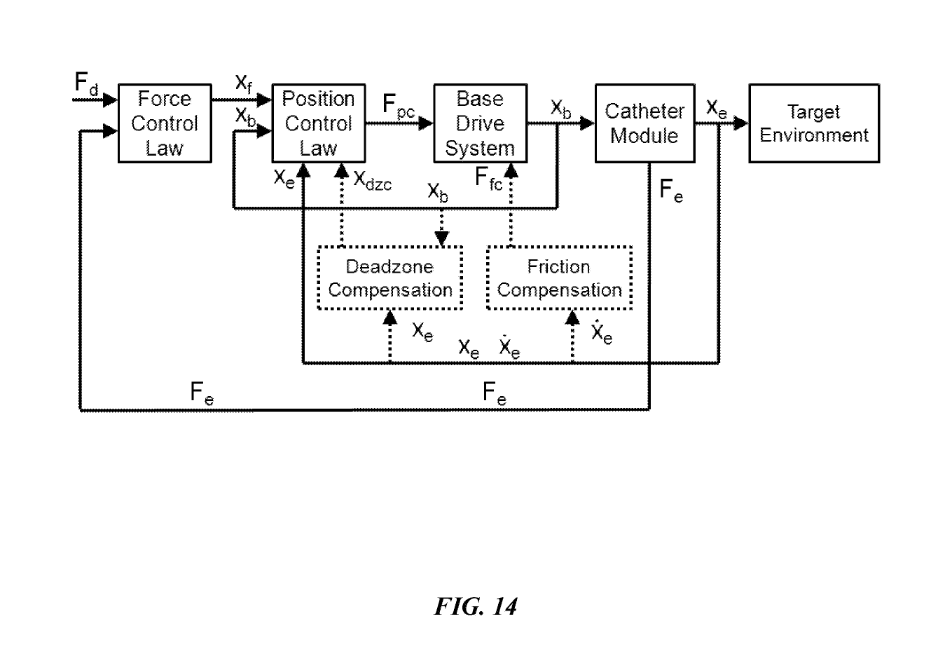

The force modulation term is

$${x}_{f} = \frac{{F}_{d}}{{K}_{e}} + {K}_{f} ( {F}_{d} - {F}_{e} ) + {K}_{fi} \int ( {F}_{d} - {F}_{e} ) d t \qquad ( 18 )$$

where K_{f} and K_{fi} are controller gains and Kₑ is the approximate stiffness of the environment, which can be estimated from tissue property values in the literature or by an on-line estimation scheme. The drive system can be controlled and commanded to follow the desired position trajectory with a standard PID controller running at 1 kHz. FIG. 14 shows a block diagram of this controller.

While the control method above improves stability over conventional force control due to the PID position controller, it does not alleviate the tracking errors caused by friction and backlash. These limitations require specific compensation methods, as indicated in the block diagram in FIG. 14 by dotted lines.

Friction compensation can be accomplished using a Coulombic friction model for the catheter and then feeds forward the friction force F_{fc}, based on an observer that predicts the velocity. The level of friction force can be determined experimentally in advance and can be a function of the catheter system design (materials, geometry) and sheath configuration (θ).

Backlash compensation can be accomplished by adding an additional term to x_{d} that adjusts the desired base position to overcome the dead zone as shown in FIG. 14. The amount of compensation, x_{dzc}, is determined using a catheter-specific dead zone model described herein.

x_{dzc}=θ(Dₛₕ−D_{gw}) (19)
where Dₛₕ is the inner diameter of the sheath and D_{gw} is the diameter of the guide-wire. The compensation term x_{dzc} is either added or subtracted from x_{d} based on the direction of target motion and the position of the guide-wire relative to the dead zone region.

In certain situations, the model-predicted dead zone width can be increased to account for the deformation of the sheath and guide-wire caused by large catheter friction resistance. In one embodiment of the invention, x_{dzc} was doubled to account for the increased dead zone width if the catheter friction was over 2 N.

In accordance with one embodiment of the invention, the catheter system included of a sheath with a 1.59 mm inner diameter and a guide-wire with a 1.50 mm outer diameter and an electromagnetic tracker (trakSTAR 1.5 mm sensor, Ascension Technology Corp., Burlington, Vt., USA, measured RMS error of 0.3 mm) was affixed to the guide-wire tip to assess control accuracy.

In this embodiment, the catheter can be fixed in a shape that roughly corresponds to the path from the femoral vein into the left atrium so that the tip was 1-2 cm from the target. In accordance with one or more embodiments of the invention, the catheter controller can be configured to perform a simple calibration routine that estimates the magnitude of the friction force and backlash dead zone and the image processing routines can be used to locate the catheter using the Radon transform algorithm and then project forward to find the target. The catheter can be served to maintain a constant distance between the catheter and tip and the target.

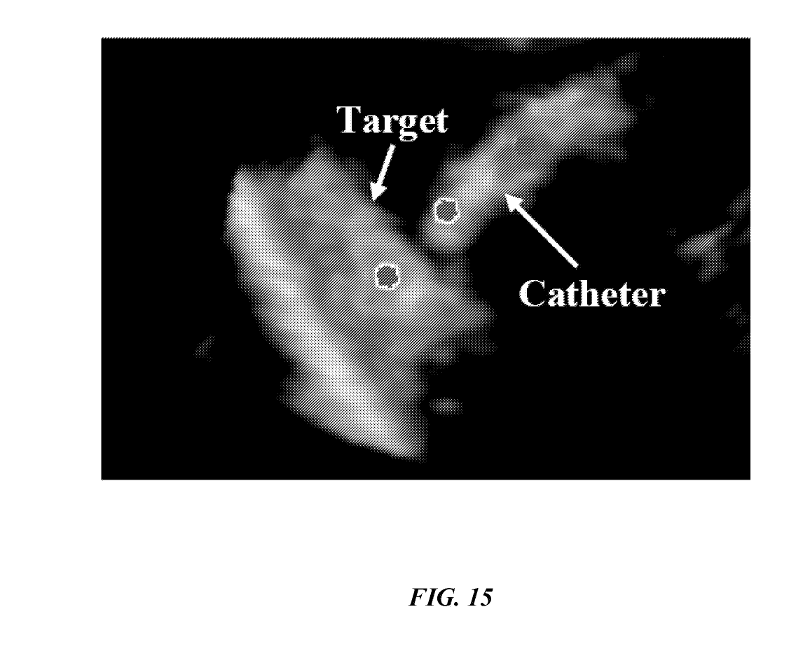

The catheter system according to the invention was successful in tracking the target. FIG. 15 shows a cross section through a typical ultrasound image volume, showing the catheter and target surface. The image includes two red dots along the catheter axis and one dot at the target, which can be placed by the image processing algorithm to permit visual confirmation of tracking during experiments.

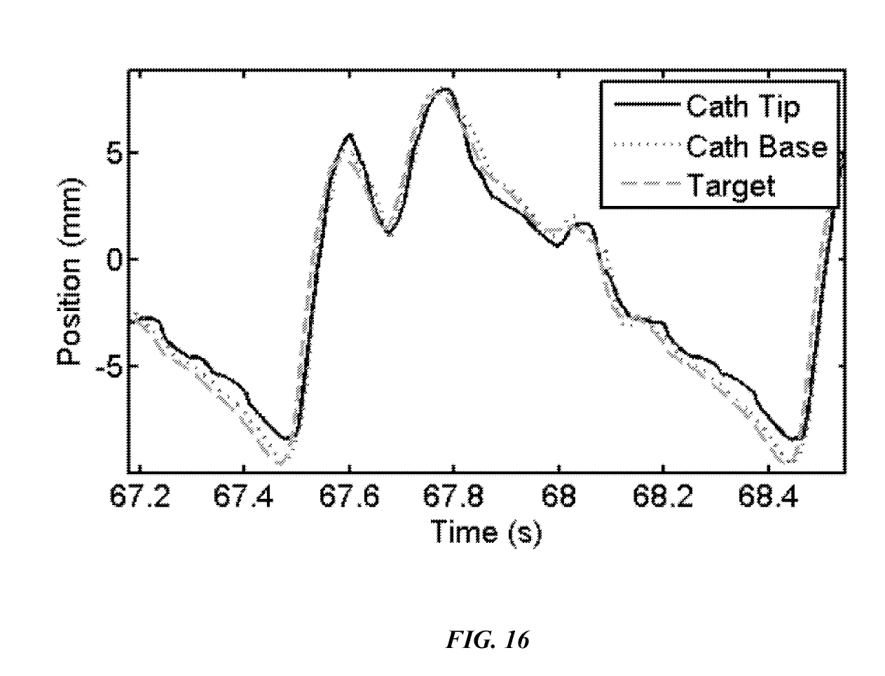

FIG. 16 shows a typical plot of catheter trajectory at both the base and tip, together with the commanded trajectory, demonstrating that the system achieves good accuracy. The RMS error between the catheter tip (measured by the electromagnetic tracker) and the actual target location was 0.86 mm. The calibration routines estimated the friction levels in the catheter to be 0.5-0.8 N and the amount of backlash to be less than 0.2 mm. In this embodiment, the main source of tracking error was friction.

In an alternative embodiment of the invention, the catheter system includes a sheath having a 1.6 mm inner diameter and a guide-wire having a 1.5 mm outer diameter. In accordance with one or more embodiments of the invention, where the gap size is small, for example, 0.1 mm, it may not be necessary to provide dead zone compensation, so only friction compensation can be provided. Further, in this embodiment seals are used at the distal end to prevent blood from flowing inside the catheter sheath, causing additional friction, resulting in friction compensation values as high 2N

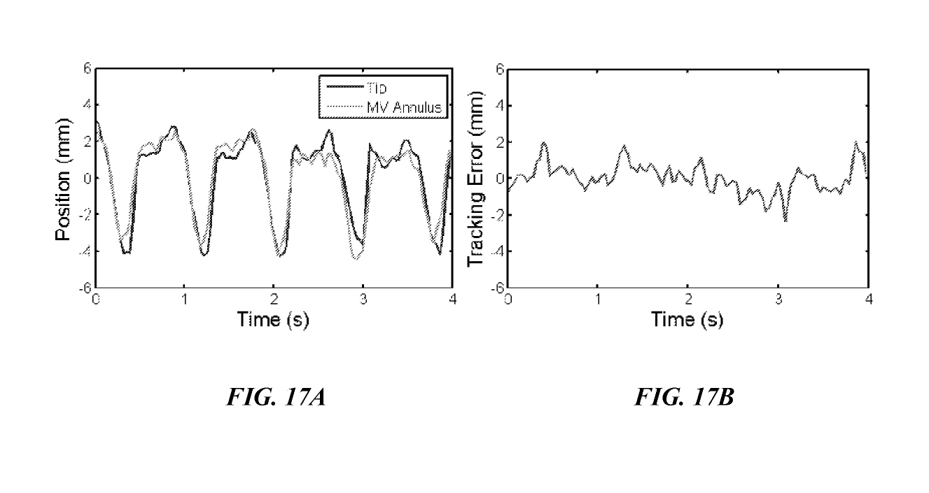

In this embodiment, the catheter system can be used to track the mitral annulus tissue of a beating heart with an RMS error of less than 1.0 mm. FIG. 17A shows the trajectory of the catheter tip and the mitral annulus. In this embodiment, tracking errors, shown in FIG. 17B, can be caused by respiration motion not captured by the tissue tracking system, performance limitation of the actuated catheter due to backlash and friction and small beat to beat variations in the heart valve motion not compensated for by the image tracking system. In alternative embodiments, tracking errors can be further reduced by configuring the tissue tracking system to measure and compensate for respiration motion of the patient or animal and compensating for beat to beat variations of the hear valve motion.

In some embodiments of the invention, the guide-wire can be hollow to allow devices or implants to be deployed from inside the guide-wire lumen. In some embodiments of the invention, the sheath can include more than one lumen in order to supply more than one guide-wire to the tip. In these embodiments, different types of guide-wires can be used to provide additional services or functions. For example, a second guide-wire can carry an imaging system, such as ICE or m-mode imaging system that can be used either by itself or in combination with an external imaging system to generate images and image data used for motion compensation.

In alternate embodiments of the invention, the imaging system 130 can be a 3D ultrasound system, a magnetic resonance imaging (MRI) system, an X-ray computed tomography (CT) system, an electromagnetic tracking system, an infrared imaging system, an m-mode ultrasound system, a 2D ultrasound imaging system, and a range of x-ray technologies including fluoroscopy. In some embodiments, one or more of these systems can be combined in order take advantage of the known benefits of some systems as compared to others, for example, with respect to different operating environments. In some embodiments of the invention, imaging system 130 can be replace by a tracking system that is cable of tracking the position of the catheter and the tissue in real time to provide suitable compensation and correction. These sensing methods can be used to track the catheter tip, the tissue target, or image and track both the tool and the target. For example, MRI, CT, and ultrasound could track both the catheter tip and the target tissue, however m-mode ultrasound could only track the tissue position and EM tracking could provide position information for only the catheter tool.

In alternative embodiments of the invention, the catheter can be provided with multiple degrees of freedom of movement. Additional motors or actuators, control systems and guide wires can be provided to enable the tip of the catheter to move in multiple directions (3 dimensions) at the same time and provide for more complex movements. In these embodiments, steering wires, like the guide-wire, can be used to control the movement of the catheter sheath at the remote end in order to position and move the tip. The steering wires can be connected to one or more motors or actuators, such as a linear actuator, to move the steering wires relative to the catheter sheath in order to move the remove end of the sheath and steer the tip. The guide-wire can be coupled to rotary motor or actuator that can be used to rotate the end effector about the axis of the guide-wire. A rotary coupling can be used to connect the guide-wire to the linear actuator. In some embodiments of the invention, the tip of the catheter can have three or more degrees of freedom, for example, by providing additional guide wire(s) connected to the tip or to the sheath control the orientation and motion of the tip. Additional actuators, either using one or more additional guide wires, or actuators attached to the tip, can be provided to support additional motion, such as tip rotation or end-effector manipulation, for example, for stitching or stapling.

In alternative embodiments of the invention, the motors and/or actuators can be located along the length of the catheter, including inside or outside the catheter sheath and either inside or outside of the body. In some embodiments, the motors and/or actuators can be located at or mounted on the tip. In addition to conventional motors and linear actuators, other actuation technologies include miniature piezoelectric motors, piezoelectric bending mechanisms, pneumatics, hydraulics, and non-conventional actuator technologies, such as shape-memory devices and combinations of technologies described.

In alternative embodiments of the invention, the catheter system can include one or more force sensors 114, at the base, and 126A, at the tip, or anywhere in between, to provide force sensing and optional force sensing control. Alternatively, force sensing can be accomplished using imaging information to sense velocity, acceleration and deformation of the catheter tip and/or the target tissue. Force sensing can be used to control the interaction between the tip and the target tissue as the tip comes in contact with the target tissue for treatment. For example, a force sensor can be used to control the tip in order to maintain a predefined constant force or varying force profile on the target tissue, while tissue is in motion. This can be used to hold the target tissue in a specific orientation while it continues to move to allow another device, such as motion compensated device to tack, stable or suture the target tissue. Alternatively, the system can be used to hold a prosthesis in place while another device tacks, staples or sutures it in place.

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

One of the aspects of the invention that differentiates it from other robotic catheters systems is that the present invention can track tissue at speeds greater than can be achieved with manual motions. This allows the robotic catheter according to the invention to track fast-moving in-cardiac structures, like the mitral annulus. For example, catheter systems according to the invention are fast enough to provide the velocities and accelerations required to track the human mitral valve annulus motions: 210 mm/s and 3800 mm/s².

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

Further, while the description above refers to the invention, the description may include more than one invention.

Additional information about the invention can be found in Appendix A, attached hereto. Each of the References cited in Appendix A documents are hereby incorporated by reference in their entirety.

What is claimed is:

1. A catheter based system comprising:
a catheter having a catheter guide-wire within a catheter sheath;
an actuator coupled to the guide-wire for moving the catheter guide-wire in the catheter sheath;
an end effector coupled to a remote end of the guide-wire;
a catheter control system connected to the actuator and adapted to control the actuator;
a location sensing system for sensing target tissue and the end effector locations and providing location data to the catheter control system; and
wherein the catheter control system controls the actuator as a function of the location data received from the location sensing system.

2. A catheter base surgical system according to claim 1 further comprising a position sensor coupled to the actuator and providing position data to the catheter control system and the catheter control system controls the motion of the guide-wire as a function of the position data.

3. A catheter based surgical system according to claim 1 wherein the location sensing system includes an ultrasound system and provides image data and location data to the catheter control system and the catheter control system controls the motion of the guide-wire as a function of at least one of the image data and the location data.

4. A catheter based surgical system according to claim 1 wherein the location sensing system is a three dimensional imaging system and provides image data and location data to the catheter control system and the catheter control system controls the motion of the guide-wire as a function of at least one of the image data and the location data.

5. A catheter based surgical system according to claim 1 further comprising a force sensor coupled between the actuator and the guide-wire, the force sensor being connected to the catheter control system to provide force data to the catheter control system, wherein the catheter control system controls the motion of the guide-wire as a function of the force data.

6. A catheter based surgical system according to claim 5 wherein the catheter control system uses the force data to determine a measure of a friction force resisting the motion of the catheter guide-wire in the catheter.

7. A catheter based surgical system according to claim 1 further comprising a force sensor coupled between the end effector and the guide-wire, the force sensor being connected to the catheter control system to provide force data to the catheter control system, wherein the catheter control system controls the motion of the guide-wire as a function of the force data.

8. A catheter based surgical system according to claim 7 wherein the catheter control system uses the force data to determine a measure of force applied to the target tissue by the end effector.

9. A catheter based surgical system according to claim 8 wherein the catheter control system uses the force data to control the motion of the end effector to provide a constant force on the target tissue.

10. A catheter based surgical system according to claim 8 wherein the catheter control system uses the force data to control the motion of the end effector to apply a variable force on the target tissue.

11. A catheter based surgical system according to claim 8 wherein the catheter control system uses the force data to control the motion of the end effector to apply a variable force on the target tissue according to a predefined force profile.

12. A catheter based surgical system according to claim 1 wherein the catheter includes a movable catheter tip and the catheter system further comprises a catheter tip position and trajectory sensor.

13. A method for controlling a catheter system comprising:
providing a catheter having a catheter guide-wire within a catheter sheath;
providing an actuator, coupled to the guide-wire, for moving the catheter guide-wire within the catheter sheath;
providing an end effector, coupled to a remote end of the guide-wire;
receiving image data of target tissue and the end effector of the catheter; and
controlling the actuator as a function of the image data.

14. A method of controlling a catheter system according to claim 13 further comprising:
providing a position sensor coupled to the actuator, the position sensor generating position data;
providing the position data to the catheter control system; and
controlling the motion of the guide-wire as a function of the position data.

15. A method of controlling a catheter system according to claim 13 further comprising:
providing a force sensor coupled between the actuator and the guide-wire for generating force data and controlling the motion of the guide-wire as a function of the three dimensional image data and the force data.

16. A method of controlling a catheter system according to claim 15 further comprising using the force data to determine a measure of a friction force resisting the motion of the catheter guide-wire in the catheter.

17. A method of controlling a catheter system according to claim 13 further comprising:
providing a force sensor coupled between the end effector and the guide-wire for generating force data and controlling the motion of the guide-wire as a function of the three dimensional image data and the force data.

18. A method of controlling a catheter system according to claim 17 further comprising using the force data to determine a measure of force applied to the target tissue by the end effector.

19. A method of controlling a catheter system according to claim 18 wherein the catheter control system uses the force data to control the motion of the end effector to provide a constant force on the target tissue.

20. A method of controlling a catheter system according to claim 18 wherein the catheter control system uses the force data to control the motion of the end effector to provide a variable force on the target tissue.

21. A method of controlling a catheter system according to claim 18 wherein the catheter control system uses the force data to control the motion of the end effector to provide a variable force on the target tissue according to a predefined force profile.

22. A method of controlling a catheter system according to claim 13 wherein the image data is two dimensional image data.

23. A method of controlling a catheter system according to claim 13 wherein the image data is three dimensional image data.

24. A method of controlling a catheter system according to claim 13 wherein the image data is ultrasound image data.
